(19) United States
(12) Patent Application Publication
(10) Pub. No.: US 2026/0035174 A1
(43) Pub. Date: Feb. 5, 2026

(54) Waste container and method for using the same

(71) Applicant: FrostPharma AB, DANDERYD (SE)

(72) Inventors: Peter WALL, Eskilstuna (SE); Henrik ALFREDSSON, BROMMA (SE)

(21) Appl. No.: 19/353,645

(22) Filed: Oct. 9, 2025

Related U.S. Application Data
(63) Continuation of application No. PCT/SE2024/050350, filed on Dec. 4, 2024.

(30) Foreign Application Priority Data
Apr. 13, 2023 (SE) 2350431-9

(51) Int. Cl. B65F 1/16; A61B 17/3217; A61M 5/32

(52) U.S. Cl. CPC B65F 1/1615; A61B 17/3217; A61M 5/3278; B65F 1/1607; B65F 2001/1692; B65F 2250/105; B65F 2250/114; B65F 2250/128

(57) ABSTRACT

A waste container has a first part having a first opening, and a second part. Fastening means are arranged to fasten and seal the second part to the first opening. Sealing surfaces together provide a radial press seal around the first opening. A snap lock of the cooperating fastening means is activated by the second part moving in a first direction in relation to the first part into the fastened position. Depth-controlling elements limit the movement of the second part in relation to the first part in the first direction beyond the point of activation. The first part and the second cap part are both made from a plastic material comprising at least 30% by volume wooden fibers. The use of such a waste container is also described.

### TECHNICAL FIELD

The embodiments of the present invention described herein relate to a waste container and to a method for using the same. Such a container can be for disposing needles and other sharp waste (a so-called “sharps container”), or for other clinical/medical waste. In particular, the waste container can be a waste container for sharps and infectious waste, biological waste, cytostatic and pharmaceutical contaminated waste, chemical waste and/or similar waste.

### BACKGROUND

Clinical or medical waste, such as used syringe needles, constitute a hazard and needs to be handled according to certain safe protocols. Normally, hospital personnel and other caregivers put such waste material in dedicated containers, in the form of lidded buckets or baskets, for transport to a facility and subsequent destruction.

Normally, the container is a disposable container, intended for one-time use only.

A number of requirements are put on such containers:
They need to be sufficiently resistant to externally applied abrasive wear and violent force, and also so that disposed waste in the container does not run the risk of protruding out from the container.
They need to be stackable in several stories without breaking.
They need to be available in standard volume sizes.
They need to comprise a lid that can be removed but that will not come off by accident during handling.
They need to provide an interface (such as a needle or blade disconnector) allowing caregivers to dispose of syringe needles and other medical waste material quickly and safely using only one hand. This may include disposing of such a needle by holding the syringe and twisting or turning it, using the lid as a resistance, so that the needle comes loose, in turn subjecting the lid to bending forces that it must be able to withstand.
They need to be sealable both temporarily and permanently.
They need to be completely self-contained, meaning that no loose parts should be present that can be lost during handling.
They need to be manufacturable in a simple process having high throughput.

Since such containers are manufactured in large volumes, it is important that the cost of producing them is low.

Conventionally, such containers are manufactured from plastic material, such as polypropylene. This is an inexpensive material that can be injection molded to a desired shape, forming a durable container.

However, in order to lower the climate footprint associated with such containers, it is desirable to produce them using a material that carries lower amounts of fossil-sourced carbon. Destruction is typically via incineration, and re-use or recycling is typically not possible.

It has turned out to be difficult to achieve such a container in such material, while still fulfilling all the above requirements.

SE 2151368-4 A discloses a container manufactured from a material being a mixture of wooden fibers and a plastic material.

In addition to the above-mentioned problems with prior art containers, they can sometimes be loaded with liquid waste. Therefore, there is a need for containers that can be sufficiently liquid-tight so as to hold such liquid waste even if the container tips over.

In particular, in some applications liquid waste is allowed for disposal, whereas in other applications it is not. For sealing a container intended for liquid waste, it is possible to use a seal to provide a liquid-proof seal of a lid or similar. However, even for applications where liquid waste is not allowed, the container should be preferably still be able to hold liquid waste if it is not used according to the instructions.

Moreover, the interface should provide flexibility to the user in terms of what waste to dispose of in the container.

### SUMMARY

The various embodiments described herein solve the above described and other problems, and proposes to manufacture a waste container using a bio-composite material, especially a material of the type being a mixture of wooden fibers and a plastic material. More particularly, the present invention proposes to arrange such a waste container in specific ways so as to fulfil the requirements.

Furthermore, the various embodiments described herein pertain to a single type of container that can both be used for applications where liquid waste is allowed and in applications in which it is not, and offers a container that is recyclable.

Hence, the various embodiments described herein relate to a waste container being associated, in an upright operating orientation of the waste container, with an axial direction, a radial direction and an angular direction.

The waste container has a first part, having a first opening with a peripheral first sealing surface; a second part, having a peripheral second sealing surface, the second part being arranged to, when moved in a first axial direction in relation to the first part into a fastened position, be permanently fastened to the first part using cooperating fastening means of the first part and the second part, the second part in the fastened position covering the first opening, the second sealing surface in the fastened position being arranged to abut against the first sealing surface so that the second part seals the first opening.

The sealing surfaces are arranged to together provide a press seal around the first opening in the fastened position, a force and a counterforce of the press seal each having a non-axial component.

The cooperating fastening means are arranged to be activated as a result of the second part moving in the first axial direction in relation to the first part into the fastened position so as to permanently fasten the second part to the first part in the fastened position, the activation comprising activating a snap lock preventing the second part from moving in a second, opposite axial direction in relation to the first part from the fastened position.

The first part and the second part further comprise cooperating depth-controlling elements, arranged to limit the movement of the second part in relation to the first part in the first axial direction beyond a point at which the activation occurs.

The first part and the second part are both made from a plastic material comprising at least 30% by volume wooden fibers.

In some embodiments, the first sealing surface is a first peripheral side wall surface of the first opening and the second sealing surface is a second peripheral side wall surface of the second part, the side wall surfaces being parallel in the fastened position and being arranged to engage by the first part and the second part being axially pressed one into the other resulting in the first and second side wall surfaces being forced in opposite radial directions as a result of elastic material deformation of the material of the first and second parts due to the axial pressing.

In some embodiments, the material deformation is at least 0.02 mm.

In some embodiments, the first part is a lower basket part and the second part is an upper lid part of the waste container.

In some embodiments, the first side wall surface is an inner wall surface of the basket part.

In some embodiments, the second side wall surface is an interior wall surface of a peripheral groove in the lid part.

In some embodiments, the coopering fastening means comprise a flange engagement structure and at least two V-shaped flanges.

In some embodiments, a bend of each V-shaped flange being arranged, as the lid part is moved into the fastened position, to be brought through a corresponding constriction of the flange engagement structure to thereby compress the V-shaped flange into a compressed state of the V-shaped flange, by the V-shaped flange bending at the bend due to engagement with the constriction, and so that, when the V-shaped flange snaps back from its compressed state into a non-compressed state of the V-shaped flange as a free end of the V-shaped flange exiting from the constriction as a result of the further axial movement of the lid part into the fastened position, the free end of the V-shaped flange in its non-compressed state engages with the flange engagement structure so as to prevent the V-shaped flange from returning back through the constriction.

In some embodiments, the depth-controlling elements are arranged to come into axial abutment with each other as the lid part is moved sufficiently in the first axial direction into its fastened state for the V-shaped flange to snap into its non-compressed state, the abutment limiting a movement in the first axial direction of the lid part in relation to the basket part beyond such point of abutment.

In some embodiments, the flange engagement structure comprises a radially extending fastening flange, in turn comprising the constrictions.

In some embodiments, the constrictions are through holes through the fastening flange.

In some embodiments, the free end of the V-shaped flange in the non-compressed state is arranged to engage with a first surface of the fastening flange.

In some embodiments, the depth-controlling elements comprise an abutment member arranged to move axially into abutment with a second surface of the fastening flange as the lid part moves axially into the fastened position in relation to the basket part.

In some embodiments, the fastening flange is arranged on the basket part.

In some embodiments, the flange engagement structure comprises a radial support surface, arranged to abut directly against the V-shaped flange and to press the V-shaped flange in a direction towards which the free end of the V-shaped flange faces when the lid part is in its fastened position.

In some embodiments, the radial support surface is a side wall of the constriction.

In some embodiments, the radial support surface is arranged to, when the lid part is in its fastened position, force the V-shaped flange in a direction that causes the V-shaped flange in turn to increase the force of the cooperating sealing surfaces.

In some embodiments, each of the V-shaped flanges is provided with a reinforcement ridge at the bend, the reinforcement ridge extending in a direction perpendicularly to the bend.

In some embodiments, at least one of the V-shaped flanges is provided on the lid part.

In some embodiments, the lid part comprises a peripheral axially extending flange from which the V-shaped flange extends axially downwards to the bend and then again axially upwards.

In some embodiments, the lid part comprises a peripheral axially extending flange from which at least one of the depth-controlling elements extends axially downwards.

In some embodiments, the axially extending flange has a material thickness that is at the most 80% of a material thickness of a part of the lid part arranged adjacent to the axially extending flange and/or of a material thickness of a main flat part of the lid part, the main flat part being arranged perpendicularly to the axial direction.

In some embodiments, the lid part comprises a radially central recessed part, defined by a radially inner periphery of a ridge of the lid part, arranged to match a shape of a lower support surface of the basket part so as to allow the basket part to stack with and on top of the lid part.

In some embodiments, a general material thickness of the lid part is at the most 2 mm.

In some embodiments, a general material thickness of the basket part is smaller than the general thickness of the lid part.

In some embodiments, the basket part is made from the plastic material but comprising a larger share of wooden fibers than the lid part.

In some embodiments, the first part is an upper lid part and the second part is a lid cap part of the waste container.

In some embodiments, the first side wall surface is an inner wall surface of the first opening.

In some embodiments, the second side wall surface is an outer wall surface of a peripheral axial flange of the lid cap part.

In some embodiments, the depth-controlling elements comprise an axially lower end of the axial flange, being arranged to abut with a corresponding angularly extending and axially facing shoulder surface of the lid part.

In some embodiments, the shoulder surface is concave in a cross-section perpendicular to the angular direction. In some embodiments, the lower end of the axial flange is concave in the same cross-section.

In some embodiments, the axial flange is arranged radially outs of any bayonet thread engagement means of the cooperating fastening means.

In some embodiments, the coopering fastening means comprise cooperating bayonet thread engagement means arranged to allow the lid cap part to be threaded into its fastened position, the threading resulting in that the lid cap part moves in the first axial direction and rotates in a first angular direction in relation to the lid part.

In some embodiments, the cooperating fastening means further comprise a sawtooth-shaped element arranged to engage with a corresponding snap lock structure, the sawtooth-shaped element being arranged to snap into engagement with the snap lock structure as the lid cap part rotates into its fastened position whereby a surface of the sawtooth-shaped element that is perpendicular to the angular direction directly abuts a corresponding parallel surface of the snap lock structure so that a rotation of the lid cap part in relation to the lid part in an opposite, second angular direction is not possible.

In some embodiments, the sawtooth-shaped element is provided on the lid part and the snap lock structure is provided on the lid cap part.

In some embodiments, the cooperating fastening means, in addition to the sawtooth-shaped element, further comprises an axial protrusion arranged to interact with the snap lock structure so as to allow angular movement of the axial protrusion across the snap lock structure back and forth and to haptically define a temporarily but not permanently fastened state of lid cap part in relation to the lid part.

In some embodiments, the lid cap part in the temporarily fastened state has moved in the first axial direction in relation to the lid part sufficiently for the first and second sealing surfaces to engage so as to seal the first opening.

In some embodiments, the cooperating fastening means comprise one axial protrusion per sawtooth-shaped element.

In some embodiments, the cooperating fastening means comprise at least four, such as six, sawtooth-shaped elements, angularly distributed about the first opening.

In some embodiments, the fastening means of the lid part is arranged at a smaller radial distance as compared to the first sealing surface.

In some embodiments, the fastening means of the lid part is connected to the first sealing surface by a conical surface slanting radially inwards as viewed from above.

In some embodiments, the depth-controlling elements comprise a rotation-limiting shoulder of the cooperating bayonet thread engagement means, the rotation-limiting shoulder being arranged to limit the rotational freedom of the lid cap part in relation to the lid part when the cooperating bayonet thread engagement means engage.

In some embodiments, the waste container comprises a lid part, in turn having a lid opening,

In some embodiments, the lid opening is provided with a disconnector plate for a syringe needle or blade, the disconnector plate being connected to the rest of the lid part at only two material connection points, the material connection points being peripherally arranged at opposite sides of the lid opening, the material connection points being designed to break if the disconnector plate is turned about an axis running between the two material connection points.

In some embodiments, the disconnector plate comprises two distinct parts, arranged side by side and together covering at least 50% of the lid opening, the two distinct parts being separated by an elongated opening extending along the axis running between the two material connection points.

In some embodiments, one of the two distinct parts comprises a syringe or blade disconnector.

In some embodiments, one of the two distinct parts comprises a tamper barrier.

In some embodiments, one of the two distinct parts comprises a syringe or blade disconnector and the other of the two distinct parts comprises a tamper barrier.

In some embodiments, the tamper barrier is connected on either side to the material connection points via a respective bridge, the bridge being arranged to break when the tamper barrier is pivoted about an axis extending between the bridges.

In some embodiments, the disconnector plate is arranged with a peripheral reinforcement flange running along the entire periphery of the disconnector plate, between the material connection points.

In some embodiments, the lid part including the disconnector plate is manufactured in one connected piece by injecting molding, where all the material forming the disconnector plate is injected axially from below at the two material connection points.

Moreover, the invention relates to a method of using a waste container according to any preceding claim, the method comprising the steps
providing a basket part and a lid part of the waste container;
permanently fastening the lid part to the basket part by bringing the lid part in the first axial direction, in relation to the basket part, into a fastened position of the lid part;
inserting clinical waste into the waste container via a lid opening in the lid part;
permanently fastening a lid cap part to the lid part by bringing the lid cap part in the first axial direction, in relation to the lid part, into a fastened position of the lid cap part, thus sealing the waste container; and
disposing of the waste container.

In some embodiments, the method further comprises the step
providing an adhesive, such as silicone, to the lid part, or a gasket, so that the adhesive or gasket seals a connection between a first sealing surface of the basket part and a second sealing surface of the lid part when the lid part is brought into the fastened position of the lid part.

In some embodiments, the method further comprises the step
providing an adhesive, such as silicone, or a gasket, to the lid cap part so that the adhesive or gasket seals a connection between a first sealing surface of the lid part and a second sealing surface of the lid cap part when the lid cap part is brought into the fastened position of the lid cap part.

In some embodiments, the method further comprises the steps
temporarily fastening the lid cap part to the lid part by bringing the lid cap part in the first axial direction, in relation to the lid part, towards but not all the way to the fastened position of the lid cap part; and
releasing the lid cap part from the lid part by bringing the lid cap part in the second axial direction in relation to the lid part.

In some embodiments, the method further comprises one or more of the steps
disconnecting a syringe or blade using a disconnector plate of the lid part, disposing the disconnected syringe or blade into the waste container;
removing the disconnector plate by turning the disconnection plate about two material connection points connecting the disconnector plate to the rest of the lid part;
removing a tamper barrier of the disconnector plate by pivoting the tamper barrier until a bridge, connecting the tamper barrier to each of the material connection points, breaks.

### DESCRIPTION OF DRAWINGS

In the following, exemplifying embodiments of the invention will be described in detail, with reference to the enclosed drawings, wherein:

FIG. 1 is a top perspective view of a first basket part of a first container according to some embodiments;

FIG. 2 is a bottom perspective view of the first basket part according to some embodiments;

FIG. 3 is a detail top perspective view of a flange engagement structure of the first basket part according to some embodiments;

FIG. 4 is a detail bottom perspective view of the flange engagement structure of the first basket part according to some embodiments;

FIG. 5 is a top perspective view of a first lid part of the first container according to some embodiments;

FIG. 6 is a detail top perspective view of a lid opening and a disconnector plate of the first lid part according to some embodiments;

FIG. 7 is a bottom perspective view of the first lid part according to some embodiments;

FIG. 8 is a bottom perspective view of a series of V-shaped flanges of the first lid part according to some embodiments;

FIG. 9 is top perspective view of the series of V-shaped flanges of the first lid part according to some embodiments;

FIG. 10 is another detail top perspective view of the lid opening and disconnector plate of the first lid part according to some embodiments;

FIG. 11 is a detail bottom perspective view of the lid opening and disconnector plate of the first lid part according to some embodiments;

FIG. 12 is a top perspective view of a second lid part of the first container according to some embodiments;

FIG. 13 is a top perspective view of a first lid cap part of the first container according to some embodiments;

FIG. 14 is a bottom perspective view of the first lid cap part according to some embodiments;

FIG. 15 is a detail bottom perspective view of snap lock structure of the first lid cap part according to some embodiments;

FIG. 16 is a top perspective view of a second container with a third lid part according to some embodiments;

FIG. 17 is a top perspective view of the second container with a fourth lid part and a corresponding lid cap part according to some embodiments;

FIG. 18 is a bottom perspective view of the second container according to some embodiments;

FIG. 19 is a perspective view of a snap lock engagement between a basket part and the fourth lid part of the second container with a first cross-section removed according to some embodiments;

FIG. 20 is a perspective view of a snap lock engagement between a basket part and the fourth lid part of the second container with a second cross-section removed according to some embodiments;

FIG. 21 is a perspective view of a snap lock engagement between the fourth lid part and the corresponding lid cap part of the second container, the lid cap part being in an initial rotary position, a first cross-section being removed according to some embodiments;

FIG. 22 corresponds to FIG. 21, but with a second cross-section removed according to some embodiments;

FIG. 23 corresponds to FIG. 21, but with a third cross-section removed according to some embodiments;

FIG. 24 is a perspective view of a snap lock engagement between the fourth lid part and the corresponding lid cap part of the second container, the lid cap part being in a permanently closed rotary position, a first cross-section being removed according to some embodiments;

FIG. 25 corresponds to FIG. 24, but with a second cross-section removed according to some embodiments;

FIG. 26 corresponds to FIG. 24, but with a third cross-section removed according to some embodiments;

FIG. 27 corresponds to FIG. 26, but with the lid cap part removed according to some embodiments;

FIG. 28 is a detail view of the view shown in FIG. 24 according to some embodiments; and

FIG. 29 is a flow chart illustrating a method according to some embodiments.

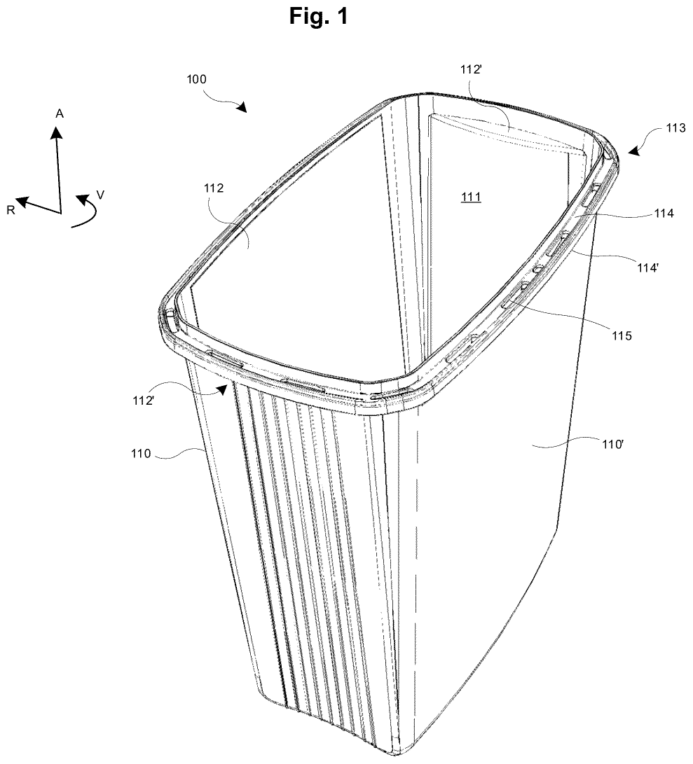

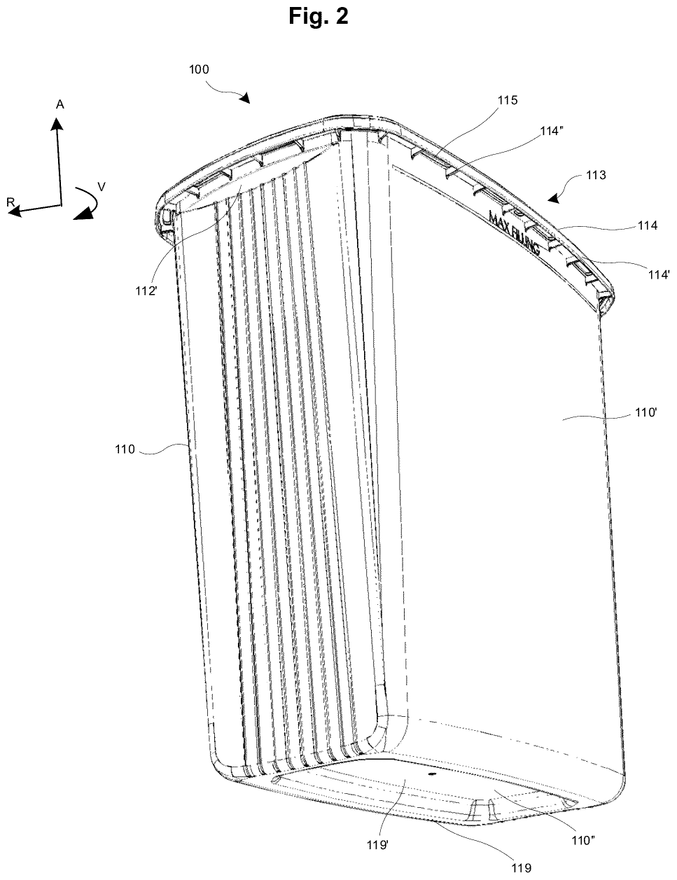

All Figures share the same reference numerals for same or corresponding parts.

### DETAILED DESCRIPTION

Hence, with general reference to the Figures, a waste container 100 is associated, in the upright operating orientation of the waste container 100, with an upwards axial direction A, an outwards radial direction R and an angular direction V. In all Figures, this upright operating orientation is shown, in which a basket opening 111 is open axially A upwards.

The waste container 100 can be a waste container for sharps and infectious waste, biological waste, cytostatic and pharmaceutical contaminated waste, chemical waste and/or similar.

The waste container 100 comprises the lower basket part 110, in turn having the upper basket opening 111. The basket part 110 also has a tapered or straight side wall 110′.

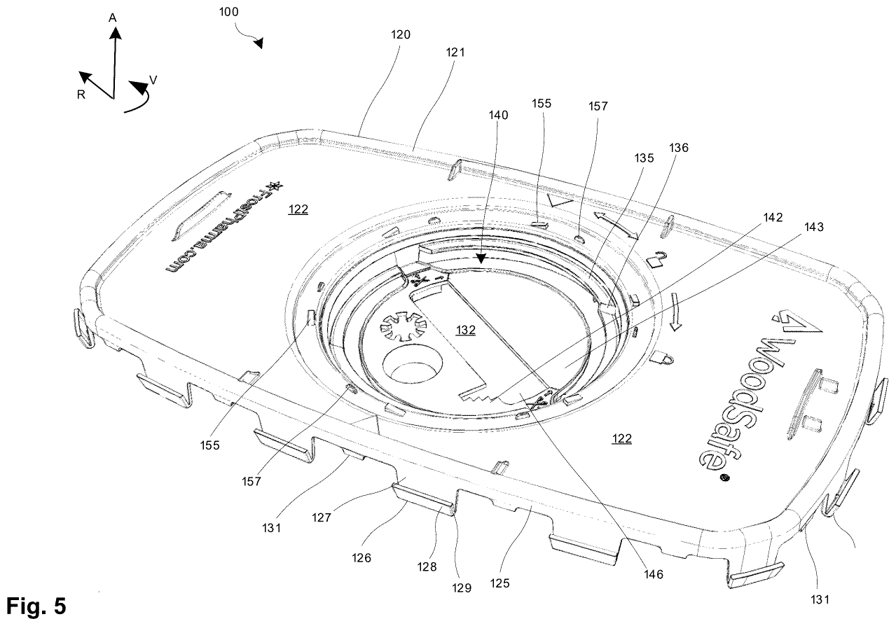

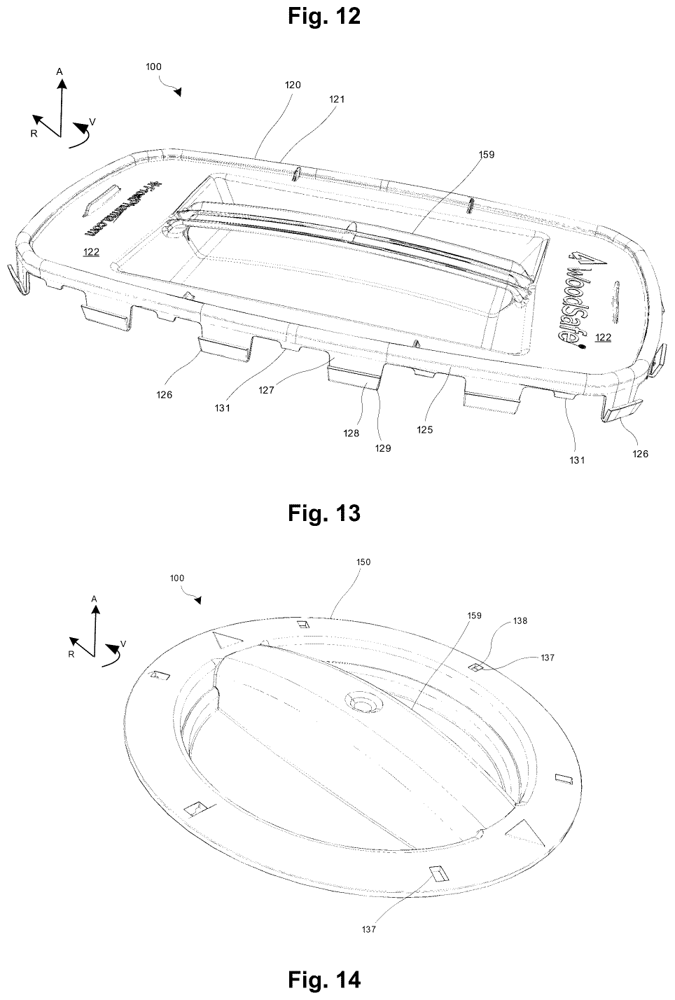

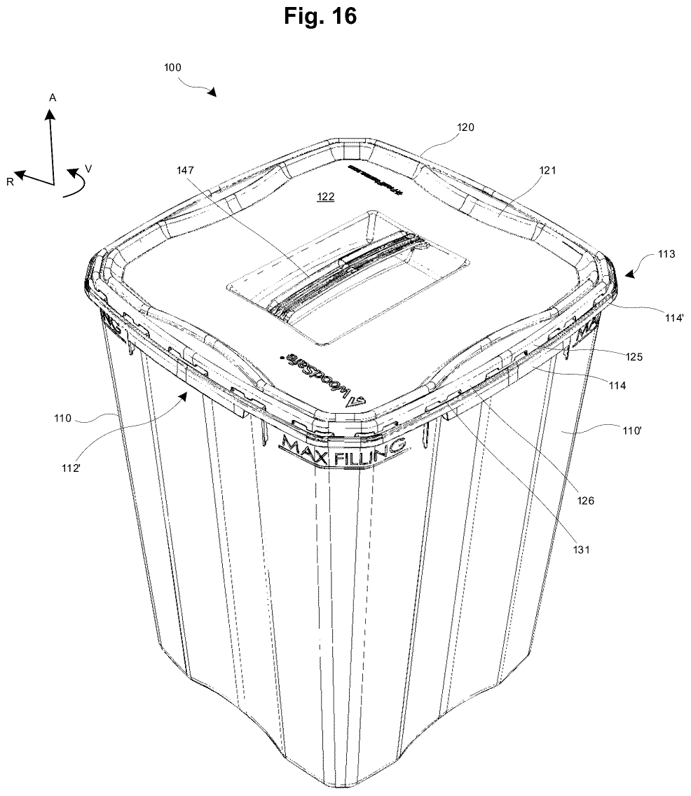

The waste container 100 also comprises an upper lid part 120 (see, for instance, FIG. 5), in turn being arranged to be fastened to the basket part 110 so as to cover and seal the basket opening 111. The lid part 120 can further comprise a lid opening 132 arranged to receive clinical waste through the lid opening 132 into the basket part 110. In other embodiments, as is shown in FIGS. 12 and 16, the lid part 120 may not have a lid opening 132 and clinical waste can instead be directly inserted via the basket opening 111. In case the lid part 120 has a lid opening 132, the waste container 100 can further comprise a lid cap part 150, arranged to cover and seal the lid opening 132.

The lid part 120 can have a handle 147, and/or the lid cap part 150 can have a handle 159.

The waste container 100 can be generally circular symmetric in a cross-section taken perpendicularly to the axial direction A, but it can also have other general shape, such as cross-sectionally square or rectangular, as the case may be. The corresponding can also apply to the lid part 120 and/or the lid cap part 150.

Everything which is the regarding the waste container 100 is equally applicable to the method of the present invention, and vice versa, as applicable.

The lid part 120 and the lid cap part 150 can both be made from a plastic material comprising at least 30% by volume wooden fibers. The lid part 120 can be made from the same material as the lid cap part 150 with same or different admixture of wooden fibers. The basket part 110 can also be made from a plastic material comprising wooden fibers, such as at least 40% by volume wooden fibers. In some embodiments, the entire container 100 is made from the same material, or at least the same plastic material with the same or different admixture of wooden fibers in its different parts 110, 120, 150.

The lid part 120, the lid cap part 150 and/or the basket part 110 can be made using injection molding of such plastic material.

The remaining material, apart from the wooden fibers, can be a conventional plastic material, such as a conventional thermoplastic, such as polypropylene, or a mixture of such plastic material with one or several other plastic materials and/or additives. More generally, the remaining material can be any suitable polymer, such as a virgin fossil-based, a recycled fossil-based or a biobased polymer. It is realized that many different such polymers can be suitable for a container of the present type, acting as reinforcement and providing sufficiently elastic, strong and abrasive-resistant properties to the composite formed when admixing wooden fibers.

Hence, the material used to manufacture the present waste container 100 can be a biocomposite material. As an example, a material of the type DuraSense®, marketed by StoraEnso, Sweden, may be used.

Using such a material, the carbon dioxide load of the waste container 100 is significantly reduced, which is advantageous from an environmental point of view.

However, such material is also significantly less elastic and more brittle as compared to, for instance, polypropylene being a material conventionally used for medical waste containers.

This makes it difficult to design such a waste container 100 that can fulfil all the initially stated requirements on waste containers. This is important, not least since they are often used to transport medical risk waste such as sharp needles and contaminated material.

The present inventors have developed a series of specific design principles that allow a waste container 100 to be manufactured using a biomaterial of the type discussed above without resulting in a quality-wise inferior product.

In the following, these design principles will be described in detail.

Regarding first material thicknesses of the parts 110, 120, 150, the lid part 120 can in some embodiments be at the most 2 mm, such as at the most 1.8 mm, such as between 1.5 and 1.7 mm, such as about 1.6 mm. The whole lid part 120 can have a substantially uniform material thickness (such as being the same or varying less than 10% across at least 50% of the total surface of the lid part 120). In some embodiments, the material thickness of the lid part 120 is constant. Hence, most or even substantially all of the lid part 120 can have a uniform material thickness. The lid cap part 150 can have a similar or identical general material thickness as the lid part 120.

To the contrary, a corresponding general material thickness of the basket part 110 can be smaller than the general thickness of the lid part 120, such as at least 0.1 or even 0.2 mm smaller. The material thickness of the basket part 110 can be less than 1.7 mm, and it may be more than 1.5 mm. An exception could be in the bottom 110″ of the basket part 110, where the material thickness can be larger, such as about 1.8 mm.

The present inventors have realized that using such general material thicknesses in the lid part 120 as compared to in the basket part 110 allows the optimization of the use of wooden fibers globally in the waste container 100 while still fulfilling the requirements given above. Specifically, the basket part 110 can be made from the plastic material comprising wooden fibers, but comprising a larger share of wooden fibers than the lid part 120 does. This means that the basket part 110 then comprises a relatively smaller amount of plastic material (such as polypropylene). Since the basket part 110 contains a larger volume of material, an overall optimized use of non-fibrous material is achieved, minimizing the carbon footprint of the entire container 100.

In the following, mechanisms for temporarily or permanently fasten and seal a first part of the container 100 to a second part of the container 100 will be described. In case the container 100 only has a basket part 110 and a lid part 120, the first part is the basket part 110 and the second part is the lid part 120. However, in case the container 100 also includes a lid cap part 150, the first part can alternatively be the lid part 120 whereas the second part is alternatively the lid cap part 150. In other words, the lid part 120 can be used to temporarily or permanently close, such as seal, the basket opening 111 of the basket part 110 and/or the lid cap part 150 can be used to temporarily or permanently close, such as seal, the lid opening 132. The general principles for achieving such temporarily or permanently closing/sealing can be partly shared across these two general cases, and therefore they are presented as two different embodiments sharing certain common features herein. It is further noted that these principles can be applied also for other lids or lid caps to cover and seal openings of waste containers of the presently presented general type. For instance, it is foreseeable that one single lid part 120 could have two separate lid openings 132, each being arranged to be covered and sealed using a separate respective lid cap part 150.

It is also specifically pointed out that the principles are useful for achieving a covering and seal of an opening in case the entity having the opening as well as the covering part are made from the material comprising wooden fibers discussed herein.

Generally, each of the first part 110 or 120 and the second part 120 or 150 have respective sealing surfaces, that are arranged to engage surface-to-surface in a sealing manner so as to achieve sealing by the second part 120 or 150 of the opening 111 or 132 of the first part 110 or 120. Such sealing can take place by the sealing surfaces being pressed together under a normal force pressure between the surfaces in question, and can involve the sealing surfaces both pressing against each other and sliding along each other up to a fastened position of the second part 120 or 150 in relation to the first part 110 or 120 where the sealing is achieved.

The sealing can be present around a full 360° periphery of the first part 120, 150 opening 111 or 132 in question.

In certain embodiments, such sealing provides complete water-tightness of the opening 111, 132 in question, and can hence provide water-tightness for the container 100 as a whole. In other embodiments, such sealing does not provide complete water-tightness, but is sufficient so as to prevent liquid enclosed inside the container 100 to leak out if the container 100 is accidentally tipped over to the side, or at least sufficient to prevent more than a smaller amount per time unit of such liquid leaking out if such accident happens. It is realized that the exact water-tightness properties will depend on the total size of the sealing surfaces that contact each other in the fastened position; the normal pressure applied between the sealing surfaces; the material properties; and so forth, and can be selected so as to achieve specific goals in terms of water-tightness.

In some embodiments, an adhesive, such as silicone, can be applied along the sealing surfaces, in direct contact with the sealing surfaces or beside the sealing surfaces, so as to achieve full watertightness. The presently described embodiments support the use of such adhesive, and can even provide a specific channel or track for application, reception and accommodation of such adhesive around the opening to be sealed. However, in some embodiments the use of such adhesive makes recycling more difficult and is therefore not preferred in such embodiments. Instead, the watertightness achieved by the sealing surfaces alone, without the use of adhesive, can be deemed adequate for such embodiments. As an alternative to the adhesive, a gasket can be used, such as a rubber gasket.

Generally, the first part 110 or 120 can have a first opening 111 or 132, in turn having a peripheral first sealing surface 112 or 133. The second part 120 or 150 can have a peripheral second sealing surface 124 or 152.

When the second part 120 or 150 is moved in a first axial A direction (downwards in the upright orientation of the exemplary containers 100 illustrated in the Figures, towards the opening to be sealed) in relation to the first part 110 or 120 into the fastened position of the first part 110 or 120, the second part 120 or 150 is arranged to, as a result of its axial A movement, be permanently fastened to the first part 110 or 120 using cooperating fastening means (see below) of the first part 110 or 120 and the second part 120 or 150.

In this fastened position of the second part 120 or 150, it is arranged to cover the first opening 111 or 132 of the first part 110 or 120. Moreover, in this fastened position, the second sealing surface 124 or 152 is arranged to abut directly against the first sealing surface 112 or 133 so that the second part 120 or 150 seals the first opening 111 or 132.

Further generally, the sealing surfaces 112 or 133; 124 or 152 can hence be arranged to together provide a press seal (press fit, interference fit) around the first opening 111 or 132 in the fastened position. In this press seal, a force as well as a resulting counterforce of the press seal can each be radial R, or at least have a non-axial A component (that is, have a radial R and/or angular V component). The force and counterforce can be a respective normal force, and hence be perpendicular to the sealing surface in question, but the press seal force can also be non-normal due to shear forces between the engaging sealing surfaces 112 or 133; 124 or 152.

In some embodiments, a non-axial A component of the press seal force and counterforce, in other words a radial R and/or angular V component, can have a magnitude of at least 70% of the total force in question. In some embodiments, the force and counterforce are predominantly radial R and/or predominantly angular V. In the specific examples shown in the Figures, the force and counterforce are both predominantly radial R.

In some embodiments, the first sealing surface is a first peripheral side wall surface 112 or 133 of the first opening 111 or 132, and the second sealing surface is a second peripheral side wall surface 124 or 152 of the second part 120 or 150. In these and other cases, the side wall surfaces 112 or 133; 124 or 152 can be parallel in the fastened position of the second part 120, 150, and can also be arranged to engage by the first part 110 or 120 being axially A pressed into the second part 120 or 150 or vice versa. This pressing then results in the first 112 or 133 and second 124 or 152 side wall surfaces being forced in opposite radial R directions as a result of local elastic material deformation of the material of the first 110 or 120 and second 120 or 150 parts due to the axial A pressing. The local material deformation can have a maximum, at or near the fastened position, which is at least 0.02 mm, or even at least 0.03 mm, for at least one but in some cases both parts 110 or 120; 120 or 150, in the latter case in opposite deformation directions.

As mentioned, the first part 110, 120 and the second part 120, 150 can comprise respective cooperating fastening means 113 or 126; 135 or 154, that will be exemplified and described below. The fastening means 113 or 126; 135 or 154 can be arranged to be activated as a result of the second part 120 or 150 moving in the first axial A direction in relation to the first part 110 or 120 into the fastened position, the activation resulting in that the second part 120 or 150 is permanently fastened to the first part 110 or 120 in the fastened position, and sealing the first part 110 or 120 opening 111 or 132 as described above.

The activation can comprise activating a snap lock, in turn preventing the second part 120 or 150 from moving back, away from the fastened position, in a second, opposite axial A direction (upwards in the exemplary containers 100 illustrated in the Figures, being in the upright operating orientation) in relation to the first part 110 or 120. Hence, as the second part 120 or 150 reaches its fastened position it achieves the above-discussed sealing of the opening 111, 132 and is permanently fastened to the first part 110, 120 in the sense that the second part 120 or 150 cannot be removed from the first part 110, 120 as any movement in the second axial A direction is prevented by the snap lock.

It is understood that the press seal itself may be arranged to allow the second part 120 or 150 to be reversibly removable, by moving the second part 120 or 150 in the second axial A direction away from the first part 110 or 120, and that it is the snap lock that can prevent such reversible removal once activate. In some embodiments, the press seal itself is continuous, in the sense that the sealing surfaces 112 or 133; 124 or 152 can move in the axial A direction by an axial A downwards force overcoming the friction between them without the sealing surface 112 or 133; 124 or 152 snapping or in any other way marking a clear point of engagement. Such point of engagement can then instead be provided by the fastening means 113 or 126; 135 or 154, possibly in combination with the below-described cooperating depth-controlling elements 117, 131, 134, 136, 151.

Moreover, the press seal itself can be non-threaded (even if it is combined with a threaded engagement provided by the fastening means 113 or 126; 135 or 154).

The first part 110 or 120 and the second part 120, 150 can furthermore be arranged so that any other relative movement between the two is also prevented. One way to achieve this is that the movement in the first axial A direction of the second part 120 or 150 in relation to the first part 110 or 120 follows a defined guide path that only allows such relative movement along the defined path. In the example provided below with respect to the fastening of the lid cap part 150 to the lid part 120, the first axial A direction movement is along a rotary R path, by screwing the lid cap part 150 axially A downwards and into a bayonet thread engagement with the lid part 120; whereas the movement in the example of fastening the lid part 120 to the basket part 110 described below is a pure downward axial A movement, lowering the lid part 120 without rotation onto the basket part 110.

Moreover, the first part 110 or 120 and the second part 120 or 150 can further comprise cooperating depth-controlling elements 117, 131, 134, 136, 151, being arranged to limit the movement of the second part 120 or 150 in relation to the first part 110 or 120 in the first axial A direction beyond a point at which the activation of the cooperating fastening means 113 or 126; 135 or 154 occurs.

This achieves that a very precise control of the relative positions of the sealing surfaces 112 or 133; 124 or 152 in the fastened position can be achieved. The snap lock can be arranged to achieve a haptic feedback to the user up its activation, so that he or she can be sure that the activation of the cooperating fastening means 113 or 126; 135 or 154 has occurred. The cooperating depth-controlling elements 117, 131, 134, 136, 151 guarantee that, in the fastened position, the relative position of the sealing surfaces 112 or 133; 124 or 152 is such so that a desired sealing is achieved of the container 100.

The present inventors have discovered that these principles can be applied to a container in which both the first part 110 or 120 and the second part 120 or 150 are made from a plastic material of the above-discussed type, comprising at least 30% by volume wooden fibers to achieve sufficient sealing without risking breaking of the material of the container 100 during handling. Specifically, the present inventors have been able to design and produce containers 100 fulfilling all stipulated requirements for waste containers, including requirements such as non-breakage when dropping the container 100 on the floor and similar. It is pointed out that this has been achieved with a container 100 having so low total weight so that the environmental benefits of admixture of wooden fiber in the production material are substantial.

Turning now specifically to the examples illustrated in the Figures of the interplay between the basket part 110 and the lid part 120, the first part in these examples is the lower basket part 110 whereas the second part is the upper lid part 120.

Then, the first side wall surface can be an inner wall surface 112 of the basket part 110, such as an upper-most end of an inner side wall surface of the basket part 110 extending down into the basket part 110, such as all the way to an interior surface of a bottom 110″ of the basket part 110. In other examples, the inner side wall surface 112 is an inside surface of an axially A extending flange being arranged radially R outside of an inside main wall surface of the basket part 110. As illustrated in the Figures, these two alternatives can be combined so that such a flange can be provided along some parts of the periphery of the basket opening 111, a connection between the main side wall of the basket part 110 and such a flange forming a grip 112′ for a user carrying the container 100.

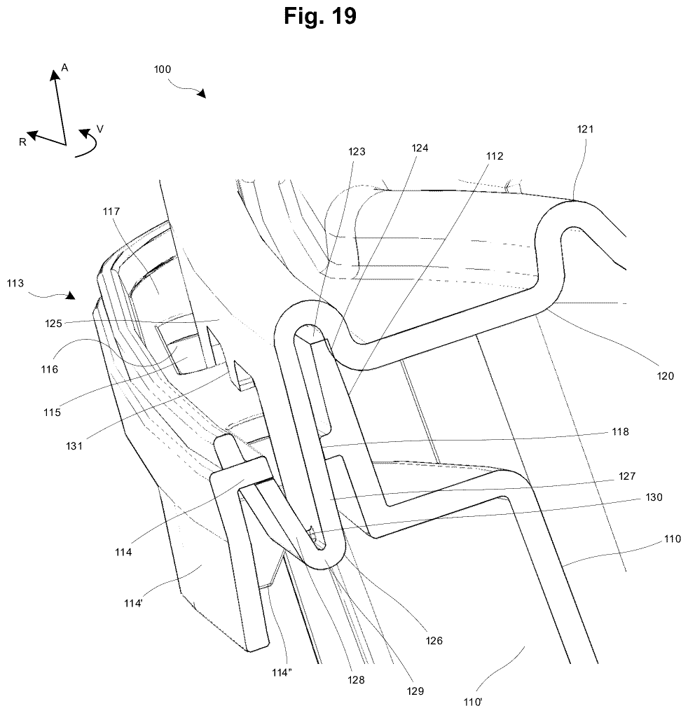

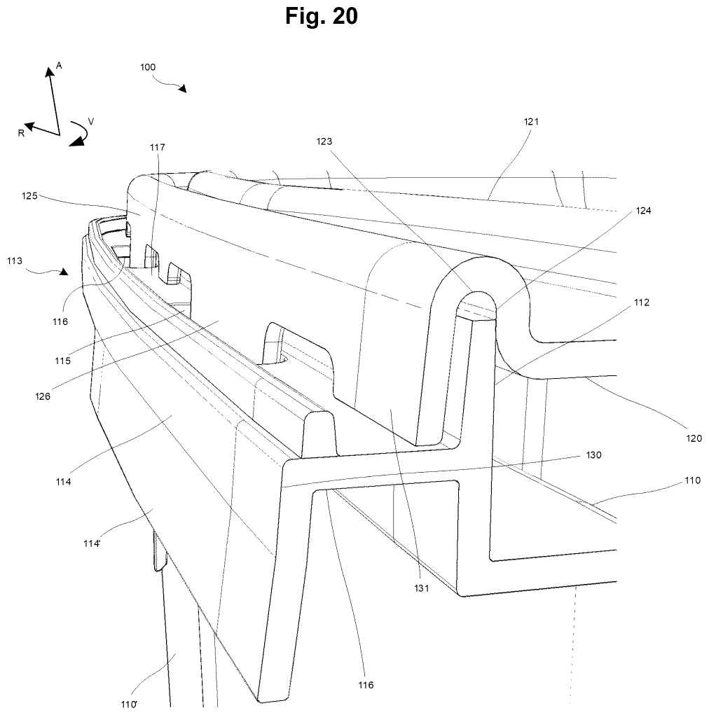

Moreover, the second side wall surface can be an interior wall surface 124 of a peripheral groove 123 in the lid part 120 (see FIGS. 19 and 20). The peripheral groove 123 can be open axially A downwards so as to be able to receive and accommodate a structure comprising the inner wall surface 112 in a way that the interior wall surface 124 can abut directly with the inner wall surface 112. This can mean that an upper end of the basket part 110 side wall, or the discussed axially A extending flange, is received into the peripheral groove 123 as the lid part 120 is axially A moved into its fastened position in relation to the basket part 110 so that the surface engagement results as a consequence.

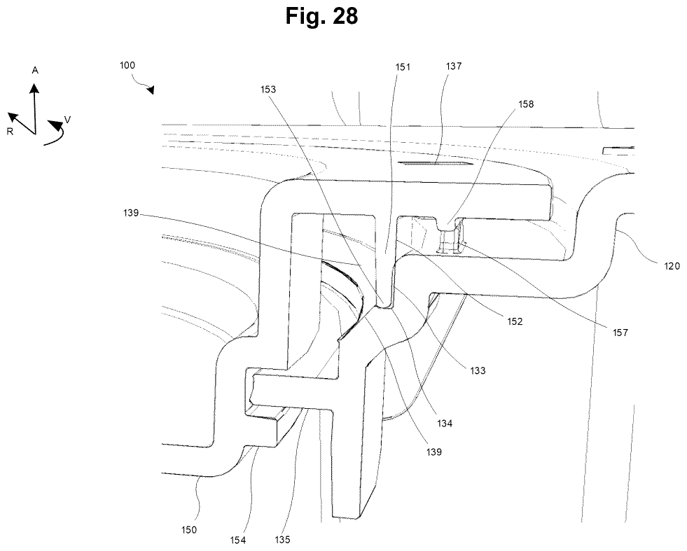

As is clear from FIGS. 19 and 28, the sealing surfaces 112 or 133; 124 or 152 are shown in their relaxed state in the drawings, not showing material deformation of the corresponding structure due to the press seal.

If an adhesive or a gasket is used, it can be applied into the peripheral groove 123 before fastening the lid part 120 to the basket part 110.

Instead of a peripheral groove 123, other structures providing at least partly radial or angular V press seal engagement between the sealing surfaces 112, 124 can be used, not being part of any groove, on the lid part 120. Such other structure can comprise a substantially axially extending surface, facing radially R inwards or outwards and/or facing any angular V direction.

It is understood that the fastening aggregate between the basket part 110 and the lid part 120, including any peripheral groove 123 and received upper edge or flange of the basket part 110, can be present along a full 360° periphery of the basket opening 111, so as to provide an adequate seal.

The coopering fastening means can comprise a flange engagement structure 113 and at least two V-shaped flanges 126. In the example shown in FIG. 5, there are sixteen V-shaped flanges 126, whereas there are twenty V-shaped flanges 126 in the example shown in FIG. 16. In some embodiments, the number of V-shaped flanges 126 can be at least four, such as at least six, or even at least eight. The V-shaped flanges 126 can be present along all sides of a polygon-shaped basket opening 111, and/or each corner of such a polygon-shaped basket opening 111 can have a respective V-shaped flange 126, and/or the V-shaped flanges 126 can be equidistantly distributed along the opening 111 periphery in the angular V direction around the basket opening 111.

The V-shaped flanges 126 can be identical or slightly different, and everything said herein regarding the V-shaped flanges 126 can in general be true for one or several of a plurality of present V-shaped flanges 126. Each V-shaped flange 126 can be at least 1 cm wide, in the angular V direction, and/or at least 1 cm axially A high.

As is perhaps best illustrated in FIGS. 19 and 20, each V-shaped flange 126 comprises a bend 129, defining the pointy end of the “V”-shape. The bend 129 in the example embodiments illustrated in the Figures points axially A downwards in the upright operating position of the container 100. The V-shaped flange 126 furthermore has a free end 128 and an opposite, connected end 127 that is integrated with or otherwise fastened to the basket part 110 or the lid part 120 (in the present example the lid part 120).

In general, the V-shaped flanges 126 can be oriented with the free end 128 facing in a radially R outwards direction, as is shown in the Figures. However, it is realized that other orientations of the free end 128 are also thinkable, such as facing in any angular V direction and/or facing radially R inwards. The present inventors have realized that the orientation illustrated in the Figures, in particular together with the below-described fastening flange 114, provides for a sturdy yet easy to use construction that can be designed to fulfil all requirements regarding durability and functionality.

The bend 129 of each V-shaped flange 126 can be arranged to, as the lid part 120 is moved axially into the fastened position, be brought through a corresponding constriction 115 of the flange engagement structure 113 to thereby compress the V-shaped flange 126 in question into a compressed state of the V-shaped flange 126, by the V-shaped flange 126 bending at the bend 129 due to engagement with the constriction 115. Hence, the V-shaped flange 126 is compressed by the constriction 115 applying, by a surface passed by the V-shaped flange 126 on its way through the constriction 115, a compressive force to the V-shaped flange 126 that compresses it elastically at the bend 129, so that an angle of the V-shape is decreased as a result of this compression. The compressive force can be at least in part applied to the free end 128 by a corresponding abutment surface of the constriction 115 abutting an outside of the free end 128 as the V-shaped flange 126 passes through the constriction 115.

It is understood that the V-shaped flange 126 can be manufactured from the same composite material as the rest of the container 100, and that it is therefore resilient but also to some degree brittle. The present inventors have concluded that the bend 129 in its relaxed state can advantageously define a bending angle of between 10° and 30°, and that the maximum compression of the V-shaped flange 126 at its passage through the constriction 115 can advantageously be between 5° and 15°.

When the V-shaped flange 126 has moved so far in the first axial A direction so that the V-shaped flange 126 is no longer under movement limitation by the constriction 115, for instance by the free end 128 no longer abutting the abutment surface, the V-shaped flange 126 snaps back from its compressed state, as a result of its resilience, into a non-compressed state of the V-shaped flange 126. Hence, the free end 128 exits from the constriction 115 as a result of the further axial A movement of the lid part 120 into the fastened position, and an angle defined by the bend 129 as a result again increases. The V-shaped flange 126 can, but does not have to, be in a relaxed state in terms of the bend 129 angle after this snapping back of the V-shaped flange 126.

However, in this non-compressed state the free end 128 can engage with the flange engagement structure 113 so as to prevent the V-shaped flange 126 from returning back through the constriction 115.

It is noted that FIGS. 19 and 20 show the non-compressed state of the V-shaped flanges 126, as the lid part 120 has been fully moved into its fastened position in relation to the basket part 110.

It is understood that the V-shaped flanges 126 are here described as belonging to the lid part 120, and the flange engagement structure 113 as belonging to the basket part 110. The present inventors have seen good results with such a design, but foresee that the opposite could potentially also be the case.

As is illustrated in FIGS. 19 and 20, the free end 128 of the V-shaped flange 126 in its non-compressed state can be arranged to engage with a first surface 116 of a fastening flange 114, the first surface 116 possibly being a surface provided on a side of the fastening flange 114 to which the free end 128 is pressed during the axial a movement into the fastened position of the lid part 120, in the Figures shows as a downwards-facing surface. The first surface 116 can be perpendicular to the axial direction A and/or can be arranged adjacent to the constriction 115.

The fastening flange 114 can be comprised in the flange engagement structure 113, and can in turn comprise the constrictions 115. Each or any constriction 115 can be a through hole through the fastening flange 114. The fastening flange 114, such as a main extension plane of the fastening plane 114, can extend in the radial R and/or angular V direction. The fastening flange 114, or the main extension plane, can be perpendicular to the axial A direction. The fastening flange 114 can be arranged on, and extend radially R outwards from, the basket part 110, such as on or from an external outside surface of the basket part 110. In some embodiments, the fastening flange 114 is at least 0.5 cm wide, or even at least 1 cm wide, in the radial R direction. In some embodiments, it is at the most 3 cm wide, or even at the most 2 cm wide, in the radial R direction.

In the example illustrated in FIGS. 19 and 20, the depth-controlling elements comprise a second surface 117 of the fastening flange 114, being arranged to abut against an abutment member 131, the abutment member 131 possibly belonging to the lid part 120. Generally, the depth-controlling elements 117, 1321 can be arranged to come into axial A abutment with each other as the lid part 120 is moved sufficiently in the first axial A direction into its fastened state for the V-shaped flange 126 to snap into its non-compressed state, this abutment then being arranged to limit a movement in the first axial A direction of the lid part 120 in relation to the basket part 110 beyond such point of abutment. The second surface 117 can be an upper surface of the fastening flange 114, at an opposite side of the fastening flange 114 to the first surface 116. Hence, as the lid part 120 is brought axially A downwards in relation to the basket part 110, the abutment member 131 will move towards direct abutment with the second surface 117 so as to prevent further axial A movement of the lid part 120 in relation to the basket part 110 once the V-shaped flanges 126 have snapped open to prevent the lid part 120 to again be lifted upwards in relation to the basket part 110. In this snapped-open state of the V-shaped flanges 126, the lid part 120 is in its fastened position, as described above.

It is specifically noted that, in this and other embodiments (such as the embodiment in which the first part is the lid part 120 and the second part is the lid cap part 150) the snap lock can be arranged to allow further movement in the first axial A direction while preventing movement in the second axial A direction. In other words, it can be the depth-controlling elements that prevent such further movement in the first axial A direction, while the snap lock only prevents movement backwards, in the second axial A direction.

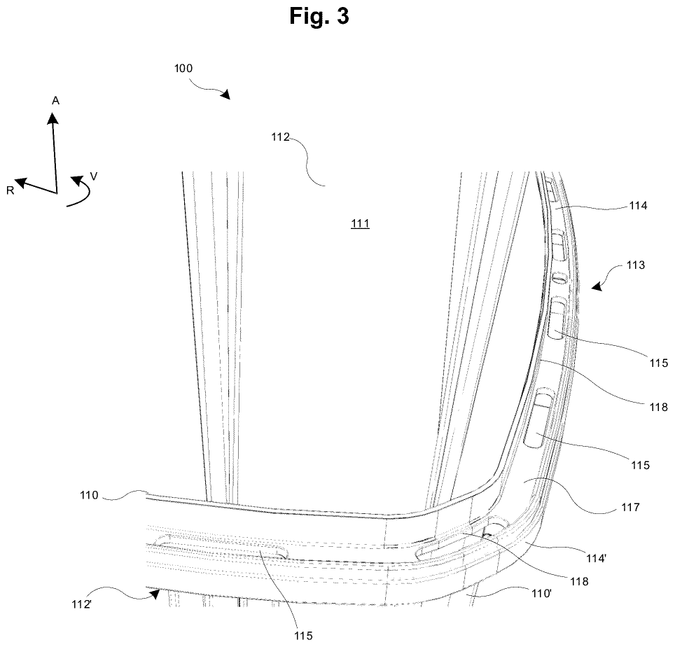

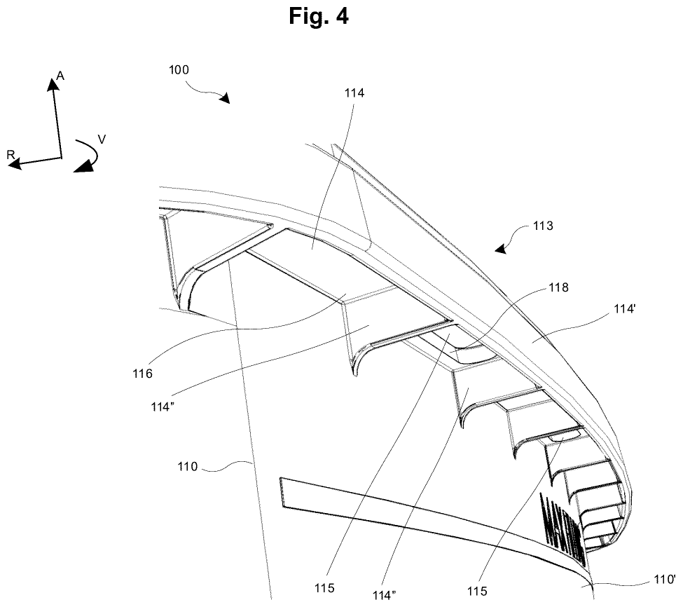

As is perhaps best illustrated in FIGS. 3 and 4, the fastening flange 114 can comprise a peripheral reinforcement flange 114′, extending in the axial A and angular V directions and running at and along a radial periphery of the fastening flange 114. This peripheral reinforcement flange 114′ can extend in the first and/or second axial A direction, such as at least 3 mm in either such axial A direction. In some embodiments, the peripheral reinforcement flange 114′ can be arranged to extend in the first axial A direction so as to completely cover the bend 129 of any V-shaped flange 126 when viewed from the radial R direction (viewed from the side in the upright operating orientation of the container 100) when the lid part 120 is in its fastened position.

Moreover, the fastening flange 114 can comprise one or more radial R reinforcement flanges 114″, extending in the axial A and radial R directions, interconnecting a main wall of the basket part 110 and the peripheral reinforcement flange 114′. The radial reinforcement flange 114″ can be at least 5 mm high in the axial A direction. In some embodiments, at least one such radial reinforcement flange 114″ is arranged between each pair of constrictions 115, and in some embodiments each constriction 115 is angularly V flanged by its own pair of such radial R reinforcement flanges 114″.

Again with specific reference to FIGS. 19 and 20, the flange engagement structure 113 can comprise a radial support surface 118, arranged to abut directly against the V-shaped flange 126 and to press the V-shaped flange 126 in a direction towards which the free end 128 of the V-shaped flange 126 faces when the lid part 120 is in its fastened position. In the example shown in FIGS. 19 and 20, this pressing is in the outwards radial R direction.

As viewed in these FIGS. 19 and 20, the radial support surface 118 is provided as a side wall of the or each constriction 115, such as a radially R inner such side wall.

In general, the radial support surface 118 can be arranged to force the V-shaped flange 126 to assume a different shape than that assumed in a relaxed state of the connected end 127 of the V-shaped flange 126 when the lid part 120 is in its fastened position. In particular, the radial support surface 118 can be arranged to, when the lid part 120 is in its fastened position, force the V-shaped flange 126 in a direction that causes the V-shaped flange 126 in turn to increase the force of the cooperating sealing surfaces 112, 124.

In the example shown in FIGS. 19 and 20, the radially R outwards pressing of the V-shaped flange 126 caused by the radial support surface 118 will bend the connected end 127 of the V-shaped flange 126 radially R outwards, in turn increasing the sealing pressure applied by the interior wall surface 124 onto the inner wall surface 112, providing a better seal. In the example shown in FIGS. 19 and 20, the force indirectly applied to the press seal between surfaces 112, 124 is applied via leverage, as the radial support surface 118 is provided at a distance from the support, located in the peripheral groove 123, about which the V-shaped flange 126 pivots. In order to achieve such an improved seal, each of the V-shaped flanges 126 can be associated with such a radial support surface 118, providing an increased sealing pressure across the full 360° of the basket opening 111.

Still referring to FIGS. 19 and 20, each of the V-shaped flanges 126 can be provided with a reinforcement ridge 130 at the bend 129, the reinforcement ridge 130 being arranged between the ends 127, 128, inside the “V”-shape, connecting an inside surface of the free end 128 to an inside surface of the connected end 127, and extending in a direction perpendicularly to the bend 129. In some embodiments, the reinforcement ridge 130 is less than 1 mm wide, such as in the axial A direction. The present inventors have discovered that such a small reinforcement ridge 130 makes it possible to manufacture the V-shaped flanges 126 with sufficient tolerances using injection molding of the above-described composite material.

As mentioned above, the V-shaped flanges 126 can be provided on the lid part 120. More specifically, the lid part 120 can comprise a peripheral axially A and angularly V extending flange 125, that in turn can be arranged to run along the full 360° periphery of the lid part 120. The V-shaped flanges 126, and more specifically the connected end 127 of the V-shaped flange 126 that is connected to the flange 125, can then extend axially A downwards, in the upright operating orientation of the container 100, to the bend 129 and then again axially A upwards, forming the free end 128.

In other words, the flange 125 then comprises the V-shaped flanges 126, being formed as protrusions from the flange 125 at intervals along the periphery of the lid part 120.

The abutment member 131, or more generally the depth-controlling element of the lid part 120, can similarly be connected to, such as form an extended part of, the flange 125, so that the abutment member 131 extends axially A downwards from the flange 125 in the upright operating orientation of the container 100.

The peripheral axially A extending flange 125 can be parallel to and/or arranged radially R inside of the peripheral reinforcement flange 114′ (if this is used) when the lid part 120 is in its fastened position. The flange 125 can comprise both V-shaped flanges 126 and abutment members 131, such as having protrusions along its periphery, the protrusions extending axially A downwards and alternatingly forming V-shaped flanges 126 and abutment members 131, as is illustrated in the Figures.

In some embodiments, one respective abutment member 131 is provided between each adjacent pair of V-shaped flanges 126 along the periphery of the lid part 120.

In some embodiments, the flange 125 has a material thickness that is at the most 80%, such as between 50% and 80%, of a material thickness of a part of the lid part 120 arranged adjacent to the flange 125 and/or of a material thickness of a main flat part of the lid part 120, such as a central recessed part 122 of the lid part 120, the main flat part in question being arranged perpendicularly to the axial A direction. The V-shaped flanges 126 and/or the abutment members 131 can have the same material thickness as the flange 125. This has been found to provide a combination of sufficient sturdiness and ease of pushing the lid part 120 into its fastened position, the V-shaped flanges 126 as a result moving through the constrictions 115 under elastic deformation. Also, this makes it possible to manufacture the lid part 120 as one single, integrated part, using injection molding with the above-discussed composite material and using only a few injection points.

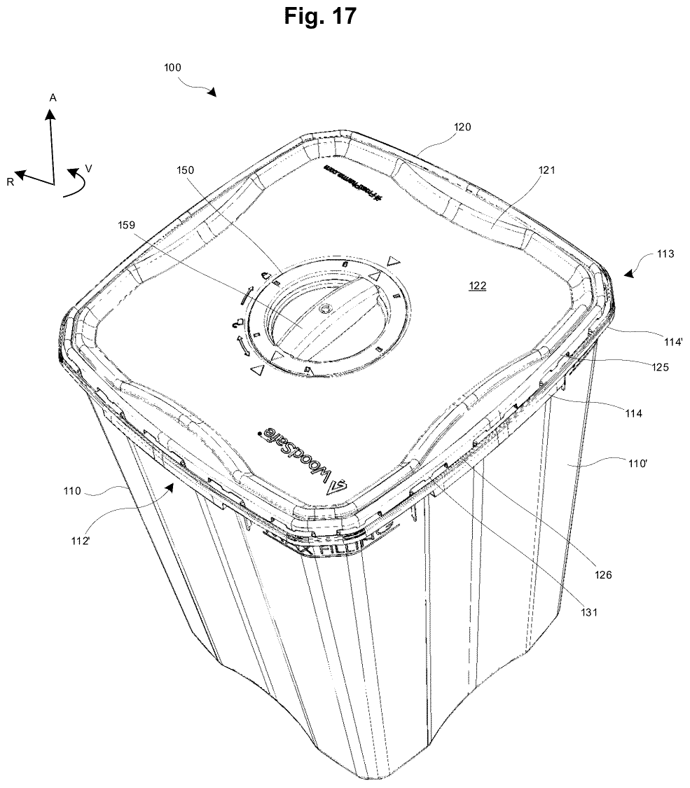

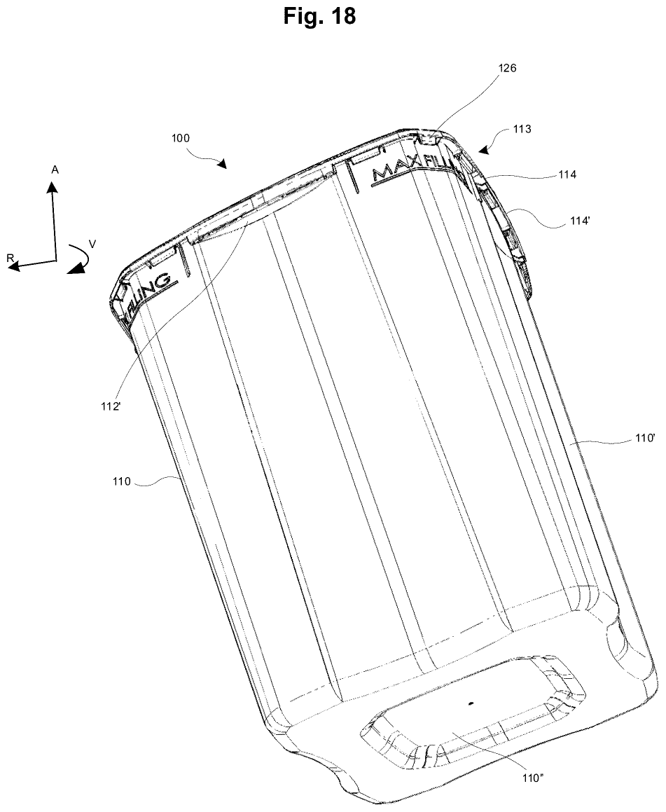

As is illustrated, for instance, in FIGS. 16 and 17, the lid part 120 can comprise the radially R central recessed part 122, defined by a radially R inner periphery of a ridge 121 of the lid part 120. The ridge 121 can be arranged radially R inside the peripheral groove 123, or alternatively be a part of the peripheral groove 123 or form the peripheral groove 123. At any rate, the ridge 121 can be arranged to match a shape of a lower support surface 119 of the basket part 110 so as to allow the basket part 110 to stack with and on top of the lid part 120. This way, two sealed containers 100 can be stacked securely on top of each other. The shape can, for instance, be symmetric along one, two or more radial R axes and be non-circular and/or non-square in a plane perpendicular to the axial A direction. The lower support surface 119 can comprise a central recession 119′, arranged to receive and accommodate the lid cap part 150 of another container 100 on top of which the basket part 110 is stacked.

As is seen in the Figures, the basket part 110 can have different shapes and sizes, and the lid part 120 will then have a corresponding shape and size so as to cover and seal the basket opening 111, that may for instance be generally square or non-square, such as generally rectangular. The present inventors foresee that the basket part 110 can be arranged to hold a total volume of at least 1 liters and/or arranged to hold a total volume of at the most 100 liters. Standard container 100 sizes (how much the basket part 110 is designed to hold as a maximum volume of clinical waste) include 6 liters, 25 liters and 50 liters.

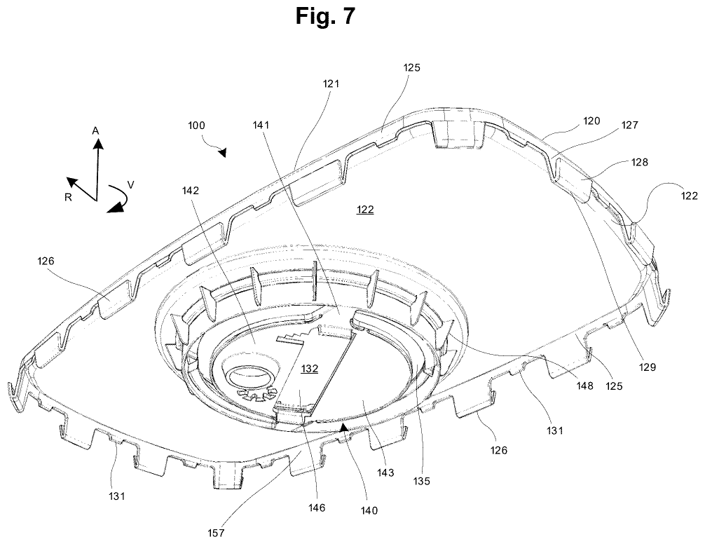

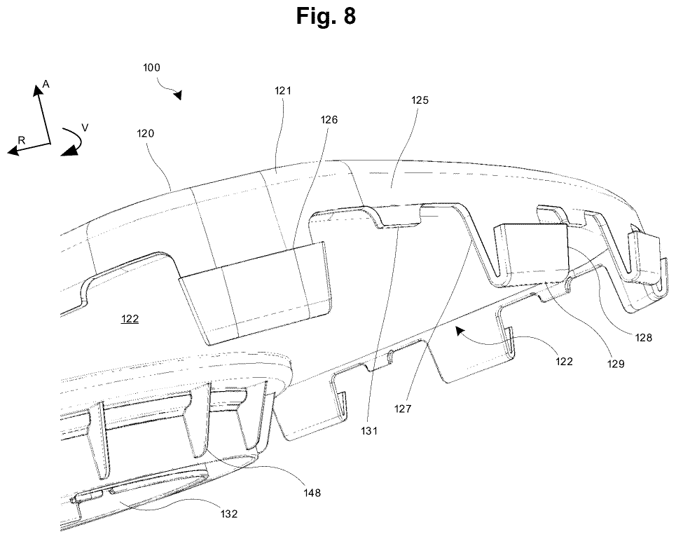

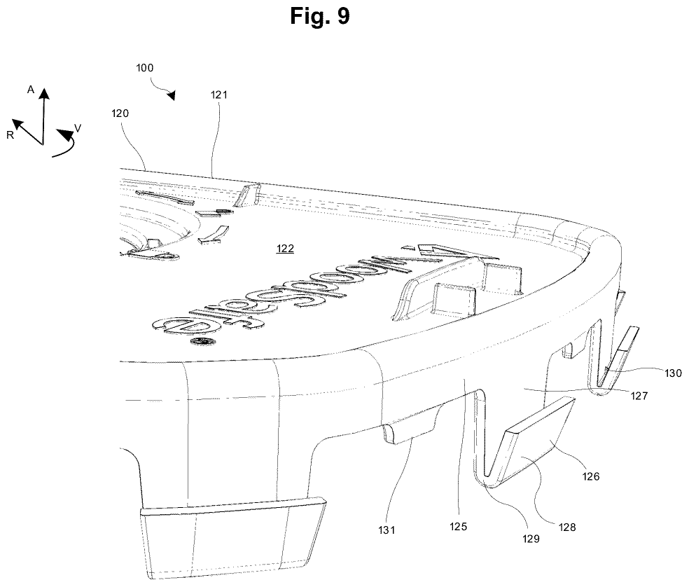

The lid part 120 can be provided with a series of axial reinforcement flanges 148 (see FIGS. 7 and 8), extending axially A downwards (in the operating orientation of the container 100) and radially R, distributed about the opening 132 so as to reinforce the opening 132. In particular, the reinforcement flanges 148 can be arranged to reinforce the material of the inner wall surface 133 so that it less easily bends radially R outwards as a result of the radial R pressure applied thereto by the flange 151 when the lid cap part 150 is in its permanently fastened position.

Above, the fastening together of the first part to the second part has been described in terms of the first part being the basket part 110 and the second part being the lid part 120. In the following, the example of the first part being the lid part 120 and the second part being the lid cap part 150 will instead be described.

It is understood that the container 100 can comprise a fastening/sealing according to the present description between the basket part 110 and the lid part 120, and/or a fastening/sealing according to the present description between the lid part 120 and the lid cap part 150. If only one of these fastening/sealing is used, the other may be provided another way, such as using a conventional sealing mechanism using a conventional snap lock or adhesive.

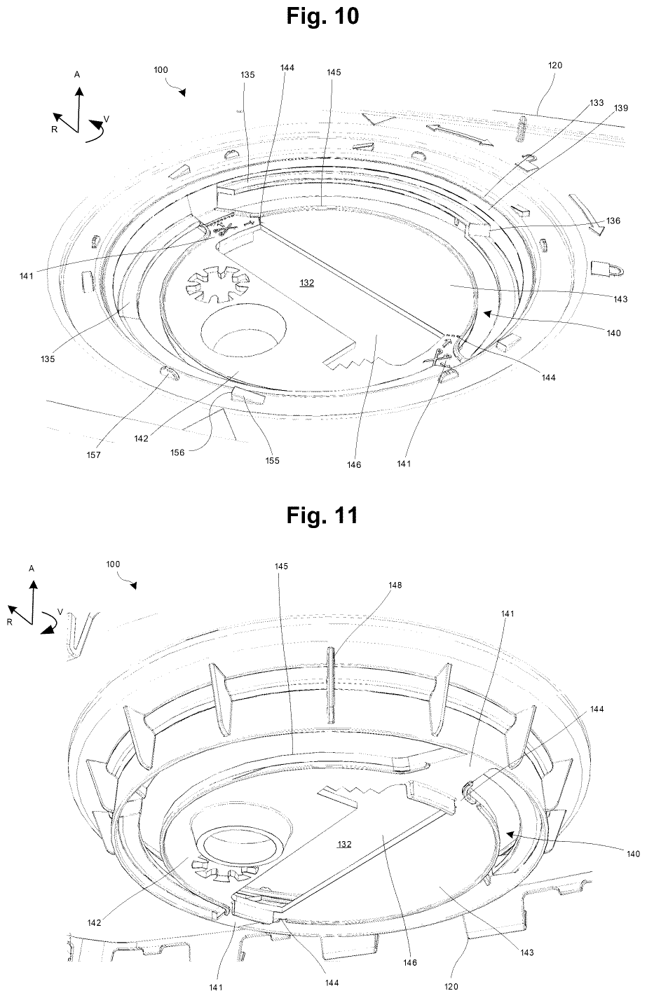

In the case where the first part is the lid part 120 and the second part is a lid cap part 150 of the waste container 100, the first side wall surface can be an inner wall surface 133 of the first opening (the lid opening) 132. See, for instance, FIG. 10. Also in this case, the second side wall surface can be an outer wall surface 152 of a peripheral axial flange 151 of the lid cap part 150. See, for instance, FIG. 14.

The axial flange 151 of the lid cap part 150 can be arranged to extend in the axial A and angular V directions, having a lower free edge and running along the whole 360° periphery of the lid cap part 150.

In this case, the depth-controlling elements between the lid part 120 and the lid cap part 150 can comprise an axially A lower end 153 of the axial flange 151, in the fastened position of the lid cap part 150 being arranged to abut directly with a corresponding angularly V extending and axially A upwards facing shoulder surface 134 of the lid part 120 (see, for instance, FIG. 28). The shoulder surface 134 can be arranged to extend along the whole 360° periphery of the lid opening 132.

In some embodiments, the shoulder surface 134 is concave in a cross-section perpendicular to the angular V direction, forming a central recess that can run along the entire 360° along the shoulder surface 134. Correspondingly, the lower axial A end 153 of the axial flange 151 can be concave in the same cross-section, for instance so as to mate with the concave shoulder surface 134 along the entire 360° about the periphery of the lid opening 132.

In some embodiments, the cooperating fastening means fastening the lid cap part 150 to the lid part 120 comprises a bayonet thread mount. In this case, the axial flange 151 can be arranged radially R outside, such as completely radially R outside, of any bayonet thread engagement means 135, 154 of the cooperating fastening means between the lid part 120 and the lid cap part 150.

Such bayonet thread mount comprises cooperating bayonet thread engagement means, for instance in the form of an angularly V slanting thread flange 135 running peripherally along the lid opening 132 of the lid part 120 and a corresponding angularly V slanting thread flange 154 running peripherally along the lid cap part 150 and arranged to allow the lid cap part 150 to be threaded into its fastened position, the threading resulting in that the lid cap part 150, when rotated into deeper threaded engagement, moves in the first axial A direction and simultaneously rotates in a first angular V direction in relation to the lid part 120 towards the fastened position of the lid cap part 150.

As used herein, the terms “bayonet thread” and “bayonet thread mount” refer to a screw mount in which the engaging threads are peripherally arranged with respect to an opening to be covered using a bayonet thread-mounted part, such as with respect to the lid opening 132 to be covered by the lid cap part 150.

The bayonet thread mount can be designed so that the lid cap part 150 is rotated a full 360°, or more, in the first angular V direction to reach its fastened position. However, the present inventors have concluded that a bayonet thread mount allowing the lid cap part 150 to reach the fastened position from a non-engaged state by rotating in the angular A direction across less than 180°, provides a sufficient seal. In similar embodiments, the lid cap part 150 to be inserted into the lid opening 132 in two or more initial rotational orientations, which can be advantageous in certain applications.

Hence, each of the slanted thread flanges 135, 154 can run across more than 360°, or less than 360°, or even less than 180°, along the periphery in question. There can be more than one of each of the thread flanges 135, 154, each providing a bayonet mount engagement between the lid part 120 and the lid cap part 150 using different angular A starting orientations of the lid cap part 150 in relation to the lid part 120.

As is illustrated in FIG. 14, the thread flanges 154 of the lid cap part 150 can comprise one or several reinforcement flanges 154′ axially A and radially R below and supporting the thread flanges 154. Since the thread flanges 154 will apply a pressing force upwards, forcing the lid cap part 150 axially A towards the lid part 120 in the fastened position of the lid cap part 150, the reinforcement flanges 154′ can help avoid breaking of the thread flanges 154 due to material failure.

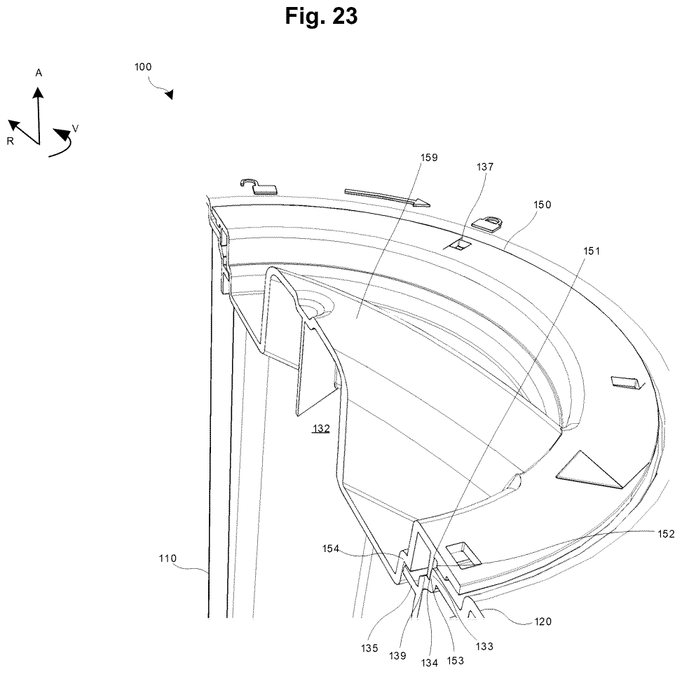

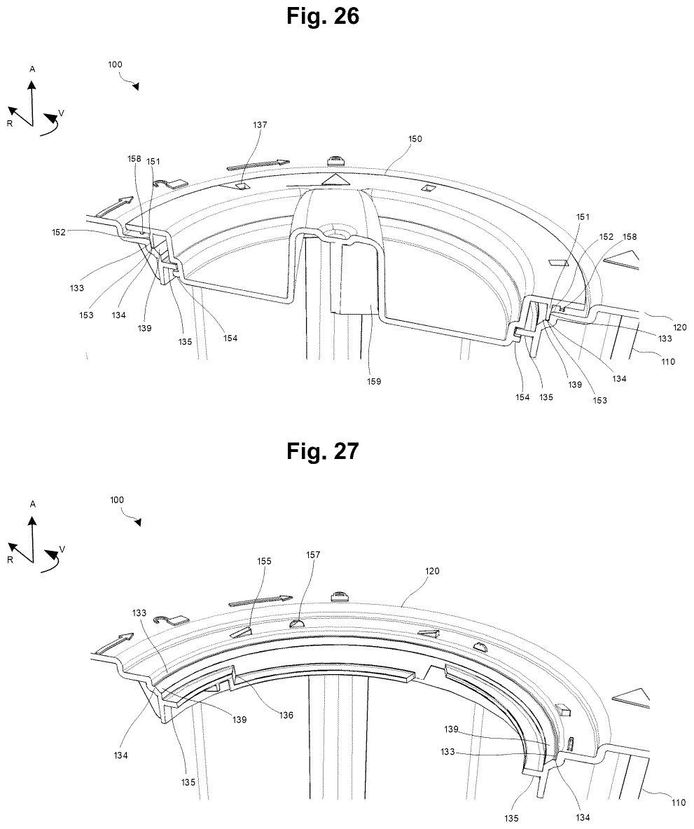

The cooperating fastening means can comprise one or several sawtooth-shaped elements 155, arranged to engage with a corresponding snap lock structure 137, each sawtooth-shaped element 155 then being arranged to snap into engagement with a corresponding snap lock structure 137 as the lid cap part 150 rotates into its fastened position whereby a surface 156 of the sawtooth-shaped element 155 that is perpendicular to the angular V direction directly abuts a corresponding parallel surface 138 of the snap lock structure 137 so that an angular V rotation of the lid cap part 150 in relation to the lid part 120 in an opposite, second angular V direction is not possible. This then means that the lid cap part 150 is permanently fastened to the lid part 120, much like is the case for the lid part 120 in relation to the basket part 110 when the V-shaped flanges 126 are fully moved through the constrictions 115 and snapped out on the other side of the constrictions 115. See, for instance, FIGS. 10, 14-15 and 21-28.

Like the snap lock of the basket part 110 and the lid part 120, the snap lock of the lid part 120 and the lid cap part 150 can allow further movement in the first axial A direction (and correspondingly also in the first angular V direction), but not the second axial A direction (and correspondingly also in the second angular V direction).

The combination of the sawtooth-shaped element 155 and the snap lock structure 137 hence corresponds to the combination of the V-shaped flange 126 and the constriction 115 having the first surface 116 as described above.

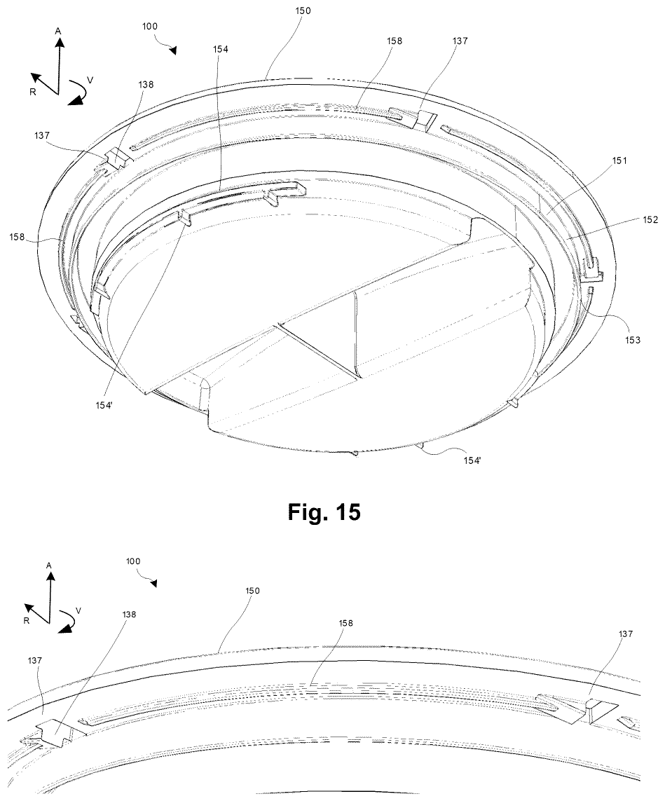

The snap lock structure 137 can also comprise, or be, a sawtooth-shaped element, arranged to engage with the sawtooth-shaped element 155 by the surface 156 engaging with a corresponding abutment surface of the snap lock structure's 137 sawtooth-shaped element. The snap lock structure 137 can comprise a hole, such as a through hole, comprising such an abutment surface. As is shown in FIG. 15, such a hole can be combined with a sawtooth-shaped element to form the snap lock structure 137.

In some embodiments, the sawtooth-shaped element 155 is provided on the lid part 120 and the snap lock structure 137 is provided on the lid cap part 150, but it is foreseen that this can also be the other way around.

As is also shown in FIG. 10, the cooperating fastening means, in addition to the sawtooth-shaped element 155 can, further comprise an axial protrusion 157, that can have slanting leading and/or trailing edges so as to form a shape having a cross-section, perpendicular to the radial direction R, without any sharp corners. The axial protrusion 157 can be arranged to interact with the snap lock structure 137 so as to, via its gently bulging cross-sectional shape, allow angular V sliding movement of the axial protrusion 157 across the snap lock structure 137 back and forth and to haptically define a temporarily but not permanently fastened state of the lid cap part 150 in relation to the lid part 120. In other words, where the sawtooth-shaped element 155 is arranged to permanently block a counter-rotation of the lid cap part 150 in relation to the lid part 120, the axial protrusion 157 will allow the lid cap part 150 to rotate across and back over a rotary location where the snap lock structure 137 passes the axial protrusion 157, the passage defining the haptic feedback.

Like the sawtooth-shaped elements 155, there can be several axial protrusions 157. Preferably, there are the same number of axial protrusions 157 as there are sawtooth-shaped elements 155, and the axial protrusions 157 are preferably angularly V distributed at the same angular V inter-protrusion 157 distance as the inter-element 155 distances used for the angular distribution of the sawtooth-shaped elements 155.

Generally, the cooperating fastening means can comprise one axial protrusion 157 per sawtooth-shaped element 155. Further generally, the cooperating fastening means can comprise at least four, such as six, sawtooth-shaped elements 155, angularly V distributed about the lid opening 132.

Using such a construction, the lid cap part 150 can be inserted into the bayonet thread engagement and turned until the haptic feedback is felt as the snap lock structure 137 arrives at or just passes one of the axial protrusions 157. Since the lid cap part 150 then engages with the lid part 120 via the bayonet thread mount, but has yet to be permanently fastened to the lid part 120, it can be described as “closed but not locked”, or temporarily fastened. This temporarily fastened rotary state of the lid cap part 150 is marked, in the embodiment shown in the Figures, using an open padlock symbol. From this position, the lid cap part 150 can be rotated in the counter (second) angular V direction out of bayonet thread engagement and hence come loose. An angular V point of entry and exit of the lid cap part 150 in relation to the lid part 120 is indicated, in the illustrated embodiment, via aligning triangles embossed in the plastic material of the parts 120, 150.

Then, as the lid cap part 150 is angularly V turned further, it reaches and passes a point at which the sawtooth-shaped element 155 reaches and passes one of the snap lock structures 137. Then, the lid cap part 150 has reached the point from which is permanently fastened to the lid part 120 and can no longer be removed without breaking the container 100. This permanently fastened rotary state of the lid cap part 150 is marked, in the embodiment shown in the Figures, using a closed padlock symbol.

It is realized that the closed state can involve simultaneous engagement between several pairs of axial protrusions 157 and snap lock structures 137; and that the permanently fastened state can involve simultaneous engagement between several pairs of sawtooth-shaped elements 155 and snap lock structures 137.

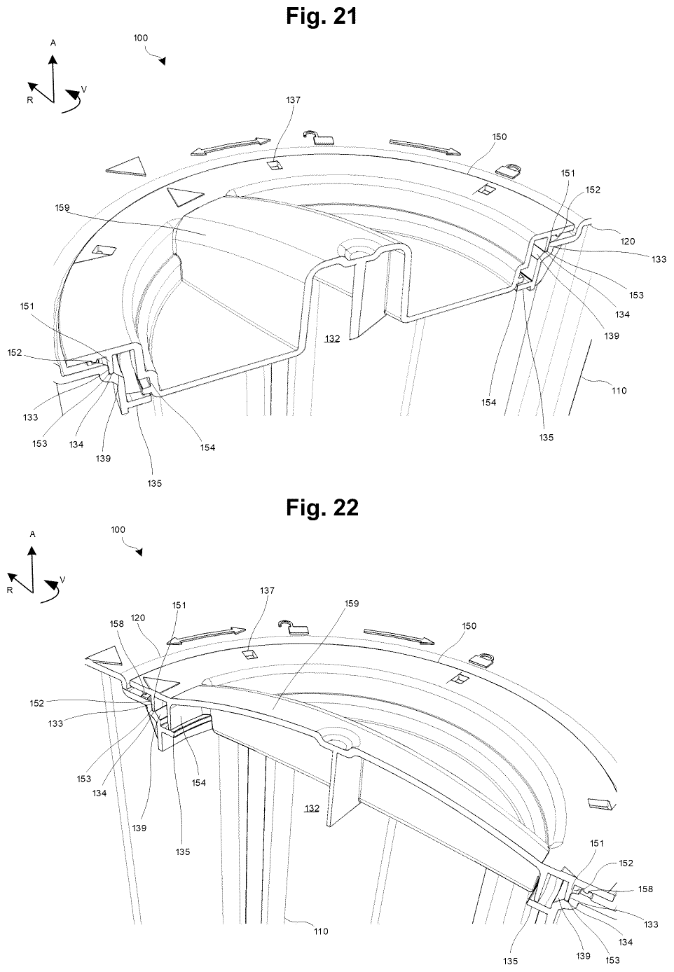

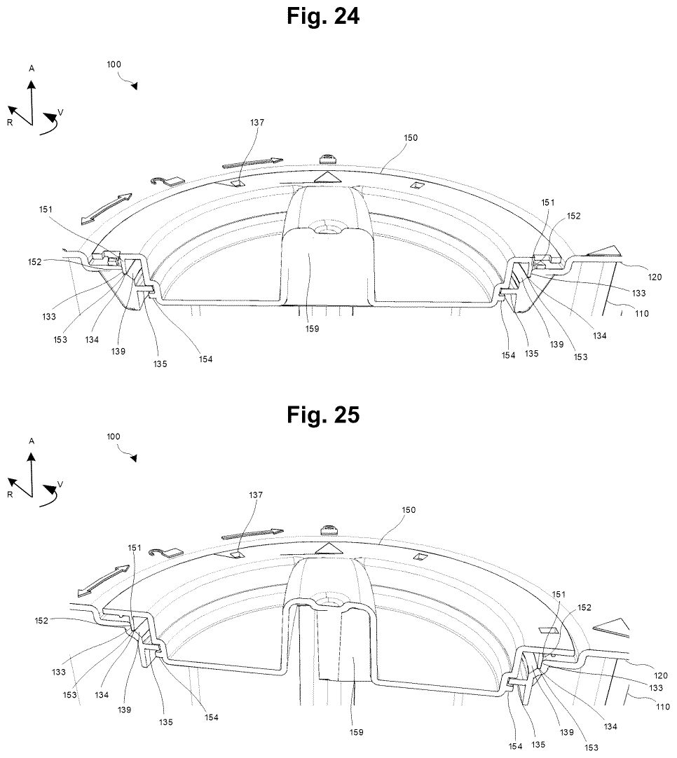

FIGS. 21-23 illustrate the state of the container 100 when the lid cap part 150 is in an original, starting position (the above-mentioned angular V point of entry and exit of the lid cap part 150 in relation to the lid part 120). FIGS. 24-28 illustrate the state of the container 100 when the lid cap part 150 has been moved into its permanently fastened position.

The snap lock structures 137 can be interconnected, such as along a whole 360° periphery of the lid cap part 150 or the lid opening 132, as the case may be, by an annular ridge 158 extending at least along angular V sections where it is not interrupted by the snap lock structure(s) 137. The ridge 158 can hence be a flange extending in the axial A and angular V directions, and can in some embodiments be less than 2 mm² in a cross-section perpendicular to the angular V direction. The axial protrusions 157 can be configured to abut against, and rest on and along, the ridge 158, providing an evenly distributed radial R pressure between the lid cap part 150 and the lid part 120 when the lid cap part 150 is in its fastened position.

In some embodiments, the lid cap part 150 in its temporarily fastened state has moved in the first axial A direction in relation to the lid part 120 sufficiently for the first 133 and second 152 sealing surfaces to engage so as to seal the first opening 132. This sealing can be sufficient to achieve complete or incomplete watertightness of the type described above, without the use of any adhesive or gasket. At any rate, such water-tightness can be achieved in the permanently fastened state of the lid cap part 150, as the lid cap part 150 will then have moved further in the first axial A direction, guided by the bayonet thread engagement, pushing surfaces 133, 152 further into mutual press seal engagement, resulting in further elastic material deformation of the lid part 120 and the lid cap part 150.

In some embodiments, the fastening means of the lid part 120, i.e. the cooperating bayonet thread mount means 135, is or are arranged at a smaller radial R distance as compared to the first side sealing surface 133. In other words, the bayonet thread mount means 135 are in this case arranged radially R inside the sealing surface 133, such as concentrically inside the sealing surface 133.

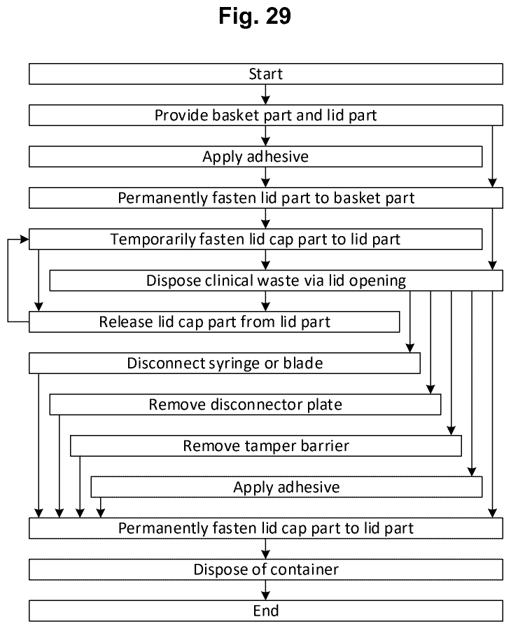

Then, the fastening means of the lid part 120, again being exemplified by the cooperating bayonet thread mount means 135, can be connected to the first sealing surface 133 by a conical surface 139, slanting radially R inwards and axially A downwards, as viewed from above. As the lid cap part 150 presses the lid part 120 material at the fastening means axially A upwards, as a result of the thread engagement deepening and the axial flange 151 abutting the shoulder surface 134, the sealing surfaces 133, 152 are as a result pressed together further, increasing the sealing of the lid cap part 150 in relation to the first opening 132. Hence, the conical surface 139 serves a similar purpose as the radial support surface 118. See FIGS. 29-32. As the support surface 118, the conical surface 139 can be arranged to operate using a leverage with respect to the increased force applied between the sealing surfaces 133, 152 as a result of the force applied to the conical surface 118 by the deepening bayonet mount engagement.

In some embodiments, the depth-controlling elements can comprise a rotation-limiting shoulder 136 of the cooperating bayonet thread engagement means 135, 154, the rotation-limiting shoulder 136 being arranged to limit the rotational freedom of the lid cap part 150 in relation to the lid part 120 when the cooperating bayonet thread engagement means 135, 154 engage. See FIG. 10, illustrating an example of such a shoulder 136, even if it is realized that such shoulder can be designed in many different ways.

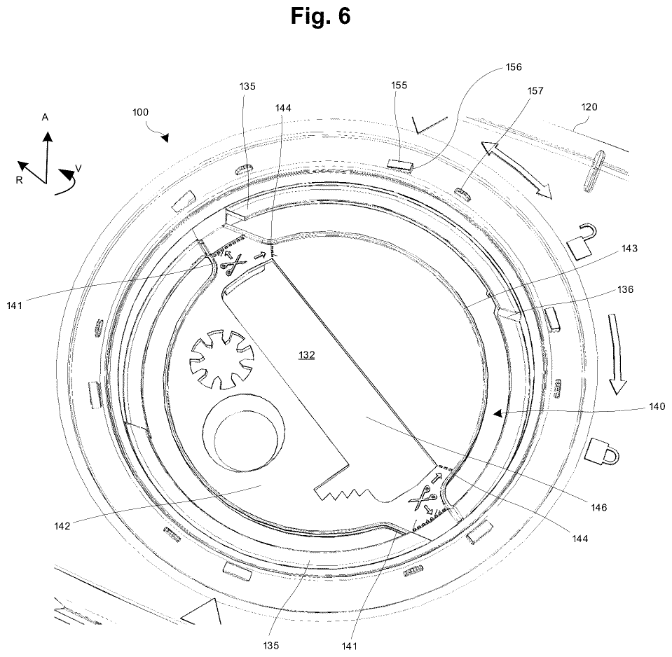

As is perhaps best shown in FIGS. 5-7, the lid opening 139 can be provided with a disconnector plate 140 for a syringe needle or blade. Such disconnector arrangements are frequently used so that users can dispose of needles and blades in a safe manner, without risking injuries or infections from such needles and blades. However, the present inventors have foreseen that, for a waste container 100 being manufactured from the above-described composite material, having a disconnector plate 140 of the type illustrated in the Figures, being connected to the rest of the lid part 120 at only two material connection points 141, the material connection points 141 being peripherally arranged at opposite sides of the lid opening 132 and the material connection points 141 and being designed to break if the disconnector plate 140 is turned about an axis running between the two material connection points 141, provides for a very flexible yet safe use.

Namely, such a disconnector plate 140 can be designed to withstand disconnection of needles and blades without breaking, whereas it is very simple and quick or a user to remove part or the whole of the disconnector plate 140 in case larger items need to be disposed of into the container 100.

In some embodiments, as in the illustrated example, any disconnector means, such as through holes or edges, used to remove blades or needles by pressing the disconnector plate 140 axially A upwards and/or downwards so as to release the blade or needle in question, are located at a distance from the axis between the material connection points 141.

The present inventors have foreseen that it is possible to arrange such a disconnector plate 140 in a lid part 120 hole 132 irrespectively of if the mechanisms described herein for temporarily and/or permanently fastening a first part to a second part are used or not. The connection points 141 can be more or less wide (in the angular V direction) and thick (in the axial A direction). They can comprise a respective local constriction of have locally straight, or substantially straight, walls, without any local constriction. What is important is that they will yield to a point where they break and the disconnector plate 140 comes loose in case the whole disconnector plate 140 is turned about the axis between the material connection points 141 (this axis normally running in the radial R direction).

Hence, the connection points 141 constitute indications of fracture in the sense that the disconnector plate 140 will break away from the rest of the lid part 120 at the connection points 141 and not elsewhere when turned in the manner. The entire disconnector plate 140, including the connection points 141, can be made as a part of the same integrated material body, such as using injection molding of the composite material, as the rest of the lid part 120.

In some embodiments, the disconnector plate 140 comprises two distinct parts, arranged side by side and together covering at least 50% of the lid opening 140 in the horizontal plane when the container 100 is in its upright operating orientation, the two distinct parts being separated by an elongated opening 146 extending along the axis running between the two material connection points 141.

One of the two distinct parts can comprise a syringe and/or blade disconnector 142. This disconnector 142 can have any shape arranged to allow a user to disconnect a syringe needle or blade without having to manually touch the needle or blade, and so that the needle or blade falls down into the interior of the container 100 after being disconnected. The Figures show an example of one possible design of the disconnector 142.

One of the two distinct parts can comprise a tamper barrier 143. The tamper barrier 143 can be primarily intended to block entry into the interior of the container 100 by the user's hand or fingers.

However, sometimes larger objects need to be disposed of into the container 100. For this purpose, the tamper barrier 143 can be arranged to be removed, so as to create a bigger through opening through the lid opening 132 into the container 100.

The tamper barrier 143 can hence be connected on either side (at both its extreme ends) to a respective one of the material connection points 141 via a respective bridge 144. The bridge 144 can be arranged to work in relation to the tamper barrier 143 in a corresponding manner as the material connection points 141 work in relation to the disconnector plate 140. Hence, the bridge 144 can be arranged to break when the tamper barrier 143 is pivoted about an axis extending between the bridges 144. As for the material connection points 141, the bridges 144 can comprise a local constriction or not comprise such a local constriction, and it will be arranged as an indication of fracture so that the tamper barrier will break away from the rest of the disconnector plate 140 by the bridges yielding until they break upon such pivoting, and the tamper barrier 143 will not break elsewhere as a consequence of the pivoting. Specifically, the bridges 144 are designed so that they will break before the material connection points 141 when the tamper barrier 143 is pivoted about the bridges 144.

It is understood that the tamper barrier 143 can also comprise disconnector means.

Hence, the user can dispose of needles and blades using the disconnector 142 without removing any part of the disconnector plate 140. The tamper barrier 143, together with the disconnector 142, prevents accidental access to the interior of the container 100 by the user's hand or fingers. The elongated opening 146 is preferably narrow enough so as not to allow a human finger to enter into the container 100, and/or wide enough to allow clinical waste to pass therethrough.

In case larger objects need to be disposed of into the container 100, the tamper barrier 143 can be quickly removed by pivoting it about the axis running between the bridges 144, while keeping the disconnector 142 in place. In case even larger objects need to be disposed of into the container 100, the entire disconnector plate 140, with or without the tamper barrier 143 depending on if the latter has already been removed, can be quickly and easily removed by pivoting the disconnector plate 140 about the material connection points 141. It is noted that these potential removals of the tamper barrier 143 or the entire connector plate 140 does not affect the ability of the lid cap part 150 to temporarily or permanently seal the container 100 as described above.

It is also noted that the construction with only two material connection points 141 connecting the disconnector plate 140 to the rest of the lid part 120, the material connection points 141 being oppositely arranged at the periphery of the lid opening 132, provides for a design of the lid part 120 that can be injection molded in one step, by arranging injection points on the underside of the lid part 120 in the upright operating orientation of the container 100, such as one injection point at either material connection point 141. Using such a design, there will be no geometric interference between the injection mold and the bayonet thread flanges 135 of the lid part 120. To achieve this, the disconnector plate 140 can also be arranged so that it is connected to the rest of the lid part 120, via the material connection points 141, axially A under (in the upright operating orientation) the bayonet thread flanges 135 of the bayonet thread mount means of the lid part 120.

In some embodiments, the disconnector plate 140 is arranged with a peripheral reinforcement flange 145 running along the entire periphery of the disconnector plate 140, between the material connection points 141.

FIG. 29 illustrates a method according to the present invention, for using a waste container 100 of the type described herein.

In a first step, the method starts.

In a subsequent step, the basket part 110 and the lid part 120 of the waste container 100 are provided.

In a subsequent step, the lid part 120 is permanently fastened to the basket part 110 as described above, by bringing the lid part 120 in the first axial A direction, in relation to the basket part 110, into the fastened position of the lid part 120. As described above, this can mean that the V-shaped flanges 126 have then fully penetrated the constrictions 115 so that the free ends 128 have snapped out and permanently locked to the first surface 116.

Before the lid part 120 is permanently fastened to the basket part 110, an adhesive, such as silicone, can be applied to the lid part 120, such as into the peripheral groove 123, so that the adhesive seals a connection between the first sealing surface 112 of the basket part 110 and the second sealing surface 124 of the lid part 120 when the lid part 120 is then brought into the permanently fastened position of the lid part 120. Such adhesives will increase the liquid-tightness of the seal, but it can also act to reinforce the seal mechanically. As an alternative to the adhesive, a gasket ca be used.

In a subsequent step, clinical waste is disposed (inserted) into the waste container 100 via the lid opening 132 in the lid part 120, such as through the opening in the lid cap part 150.

In a step that can be performed before the clinical waste material is disposed into the container 100, the lid cap part 150 can be temporarily fastened to the lid part 120, by bringing the lid cap part 150 in the first axial A direction, in relation to the lid part 120, towards but not all the way to the fastened position of the lid cap part 150. As described above, this axial A movement can also involve angular V rotation into the bayonet thread mount engagement.

Then, the lid cap part 150 can again be released from the lid part 120 by bringing the lid cap part 150 in the second, opposite, axial A direction in relation to the lid part 120, out of the bayonet thread mount engagement.

Such temporary fastening and release can be repeated one or several times, depending on usage.

Before permanently closing the container 100 by permanently fastening the lid cap part 150 to the lid part 120, one or several of the following steps can be performed one or several times:

Firstly, disconnecting a syringe or blade using the disconnector plate 140 of the lid part 120, disposing the disconnected syringe or blade into the waste container 100 (this step can be the same as the above-described step wherein the clinical waste is disposed via the lid opening);

Secondly, removing the disconnector plate 140 by turning the disconnection plate 140 about the two material connection points 141 connecting the disconnector plate 140 to the rest of the lid part 120, as described above; and

Thirdly, removing the tamper barrier 143 of the disconnector plate 140 by pivoting the tamper barrier 143 until its bridges 144, connecting the tamper barrier 143 to each of the material connection points 141, breaks.

In a subsequent step, the lid cap part 150 is then finally permanently fastened to the lid part 120 by bringing the lid cap part 150 in the first axial A direction, in relation to the lid part 120, into the permanently fastened position of the lid cap part 150, thus sealing the waste container 100. Again, this can also involve angular V rotation into the bayonet thread mount engagement, as described above.

Before this permanent fastening of the lid cap part, an adhesive, such as silicone, can be applied to the lid cap part 150 so that the adhesive seals a connection between the first sealing surface 133 of the lid part 120 and the second sealing surface 152 of the lid cap part 150 when the lid cap part 150 is brought into the fastened position of the lid cap part 150. Such adhesive can be applied on the radial inside or outside of the axial flange 151. As an alternative to the adhesive, a gasket can be used.

Again, such adhesives or gaskets will increase the liquid-tightness of the seal, but it can also act to reinforce the seal mechanically.

In a subsequent step, the waste container 100 is disposed. Such disposing preferably comprises material recycling of at least part of the container material 100. Alternatively, the container material 100 can be incinerated.

In a subsequent step, the method ends.

Above, preferred embodiments have been described. However, it is apparent to the skilled person that many modifications can be made to the disclosed embodiments without departing from the basic idea of the invention.

It is generally realized that the inventive effort underlying the present invention is to make a series of modifications to a conventional waste container so as to allow the present biocomposite to be used instead of, for instance, 100% polypropylene. Herein, a number of such specific modifications have been described, and various ways of combining such modifications. It is, however, realized that the modifications and features described herein can be combined also in other ways, as the case may be.

Everything that has been described in relation to the present container is equally applicable to the described method, and vice versa.

Hence, the invention is not limited to the described embodiments, but can be varied within the scope of the enclosed claims.

1. A waste container being associated, in an upright operating orientation of the waste container, with an axial direction (A), a radial direction (R) and an angular direction (V), the waste container comprising:
a first part having a first opening with a peripheral first sealing surface;
a second part having a peripheral second sealing surface, the second part being arranged to, when moved in a first axial (A) direction in relation to the first part into a fastened position, be permanently fastened to the first part using cooperating fastening means of the first part and the second part, the second part in the fastened position covering the first opening, the second sealing surface in the fastened position being arranged to abut against the first sealing surface so that the second part seals the first opening,
wherein:
the sealing surfaces are arranged to together provide a press seal around the first opening in the fastened position, a force and a counterforce of the press seal each having a non-axial (A) component,
the cooperating fastening means are arranged to be activated as a result of the second part moving in the first axial (A) direction in relation to the first part into the fastened position so as to permanently fasten the second part to the first part in the fastened position, the activation comprising activating a snap lock preventing the second part from moving in a second, opposite axial (A) direction in relation to the first part from the fastened position,
the first part and the second part further comprise cooperating depth-controlling elements, arranged to limit the movement of the second part in relation to the first part in the first axial (A) direction beyond a point at which the activation occurs,
the first part and the second part are both made from a plastic material comprising at least 30% by volume wooden fibers,
the first sealing surface is a first peripheral side wall surface of the first opening and the second sealing surface is a second peripheral side wall surface of the second part, the side wall surfaces being parallel in the fastened position and being arranged to engage by the first part and the second part being axially (A) pressed one into the other resulting in the first and second side wall surfaces being forced in opposite radial (R) directions as a result of elastic material deformation of the material of the first and second parts due to the axial (A) pressing, and
the material deformation is at least 0.02 mm.

2. The waste container of claim 1, wherein the first part is a lower basket part and the second part is an upper lid part of the waste container.

3. The waste container of claim 1, wherein the first side wall surface is an inner wall surface of the basket part.

4. The waste container of claim 1, wherein the second side wall surface is an interior wall surface of a peripheral groove in the lid part.

5. The waste container of claim 1, wherein:
the cooperating fastening means comprise a flange engagement structure and at least two V-shaped flanges, and
a bend of each V-shaped flange being arranged, as the lid part is moved into the fastened position, to be brought through a corresponding constriction of the flange engagement structure to thereby compress the V-shaped flange into a compressed state of the V-shaped flange, by the V-shaped flange bending at the bend due to engagement with the constriction, and so that, when the V-shaped flange snaps back from its compressed state into a non-compressed state of the V-shaped flange as a free end of the V-shaped flange exiting from the constriction as a result of the further axial (A) movement of the lid part into the fastened position, the free end of the V-shaped flange in its non-compressed state engages with the flange engagement structure so as to prevent the V-shaped flange from returning back through the constriction.

6. The waste container of claim 5, wherein the depth-controlling elements are arranged to come into axial (A) abutment with each other as the lid part is moved sufficiently in the first axial (A) direction into its fastened state for the V-shaped flange to snap into its non-compressed state, the abutment limiting a movement in the first axial (A) direction of the lid part in relation to the basket part beyond such point of abutment.

7. The waste container of claim 5, wherein the flange engagement structure comprises a radially (R) extending fastening flange, in turn comprising the constrictions.

8. The waste container of claim 7, wherein the constrictions are through holes through the fastening flange.

9. The waste container of claim 7, wherein the free end of the V-shaped flange in the non-compressed state is arranged to engage with a first surface of the fastening flange.

10. The waste container of claim 7, wherein the depth-controlling elements comprise an abutment member arranged to move axially (A) into abutment with a second surface of the fastening flange as the lid part moves axially (A) into the fastened position in relation to the basket part.

11. The waste container of claim 7, wherein the fastening flange is arranged on the basket part.

12. The waste container of claim 5, wherein the flange engagement structure comprises a radial support surface, arranged to abut directly against the V-shaped flange and to press the V-shaped flange in a direction towards which the free end of the V-shaped flange faces when the lid part is in its fastened position.

13. The waste container of claim 12, wherein the radial support surface is a side wall of the constriction.

14. The waste container of claim 12, wherein the radial support surface is arranged to, when the lid part is in its fastened position, force the V-shaped flange in a direction that causes the V-shaped flange in turn to increase the force of the cooperating sealing surfaces.

15. The waste container of claim 5, wherein each of the V-shaped flanges is provided with a reinforcement ridge at the bend, the reinforcement ridge extending in a direction perpendicularly to the bend.

16. The waste container of claim 5, wherein:
at least one of the V-shaped flanges is provided on the lid part, and
the lid part comprises a peripheral axially (A) extending flange from which the V-shaped flange extends axially (A) downwards to the bend and then again axially (A) upwards.

17. A method for using a waste container as described om claim 1, the method comprising:
providing a basket part and a lid part of the waste container;
permanently fastening the lid part to the basket part by bringing the lid part in the first axial (A) direction, in relation to the basket part, into a fastened position of the lid part;
inserting clinical waste into the waste container via a lid opening in the lid part;
permanently fastening a lid cap part to the lid part by bringing the lid cap part in the first axial (A) direction, in relation to the lid part, into a fastened position of the lid cap part, thus sealing the waste container; and
disposing of the waste container.

18. The method of claim 17, further comprising:
providing an adhesive, such as silicone, or a gasket, to the lid part so that the adhesive or gasket seals a connection between a first sealing surface of the basket part and a second sealing surface of the lid part when the lid part is brought into the fastened position of the lid part.

19. The method of claim 17, further comprising:
providing an adhesive, such as silicone, or a gasket, to the lid cap part so that the adhesive or gasket seals a connection between a first sealing surface of the lid part and a second sealing surface of the lid cap part when the lid cap part is brought into the fastened position of the lid cap part.

20. The method of claim 17, further comprising:
temporarily fastening the lid cap part to the lid part by bringing the lid cap part in the first axial (A) direction, in relation to the lid part, towards but not all the way to the fastened position of the lid cap part; and
releasing the lid cap part from the lid part by bringing the lid cap part in the second axial (A) direction in relation to the lid part.

21. The method of claim 17, further comprising one or more of:
disconnecting a syringe or blade using a disconnector plate of the lid part, disposing the disconnected syringe or blade into the waste container;
removing the disconnector plate by turning the disconnection plate about two material connection points connecting the disconnector plate to the rest of the lid part;
removing a tamper barrier of the disconnector plate by pivoting the tamper barrier until a bridge, connecting the tamper barrier to each of the material connection points, breaks.
